(19) United States
(12) Patent Application Publication
(10) Pub. No.: US 2024/0277238 A1
(43) Pub. Date: Aug. 22, 2024

(54) DISPLAY DEVICE AND METHOD OF MEASURING BLOOD PRESSURE USING THE DISPLAY DEVICE

(71) Applicant: Samsung Display Co., LTD., Yongin-si (KR)

(72) Inventor: Chul KIM, Yongin-si (KR)

(21) Appl. No.: 18/517,802

(22) Filed: Nov. 22, 2023

(30) Foreign Application Priority Data
Feb. 20, 2023 (KR) 10-2023-0022400

(51) Int. Cl. A61B 5/021; A61B 5/00

(52) U.S. Cl. CPC A61B 5/02116; A61B 5/6898; A61B 2562/0247

(57) ABSTRACT

A display device includes a first pixel which emits first light; a second pixel which emits second light having a different wavelength from the first light; a photosensor which senses the first light and the second light which are alternately provided a plurality of times; and a pulse wave sensing circuit which receives information about the first light and information about the second light sensed by the photosensor and generates a first pulse wave signal based on the information about the first light and a second pulse wave signal based on the information about the second light; and a main processor which receives the first pulse wave signal and the second pulse wave signal and calculates blood pressure information.

This application claims priority to Korean Patent Application No. 10-2023-0022400, filed on Feb. 20, 2023, and all the benefits accruing therefrom under 35 U.S.C. § 119, the content of which in its entirety is herein incorporated by reference.

### BACKGROUND

### 1. Field

The disclosure relates to a display device and a method of measuring blood pressure using the display device.

### 2. Description of the Related Art

A display device is a device for displaying a screen and used not only as a television (TV) or a monitor but also as a portable smartphone or a tablet personal computer (PC). A portable display device is typically equipped with various functions. For example, a camera and a fingerprint sensor may be provided in a portable display device to perform the functions.

With the recent spotlight on the healthcare industry, methods of obtaining biometric information about health more easily are being developed. For example, attempts are being made to change a conventional oscillometric pulse measurement device into a portable electronic device. However, the electronic pulse measurement device itself typically uses an independent light source, sensor and display and has the inconvenience of having to be carried separately. Accordingly, a display device having a built-in blood pressure measurement system is being actively developed.

A user may be requested by the display device having the built-in blood pressure measurement system to maintain an action for measuring blood pressure for a certain period of time. When the blood pressure measurement system is built in the display device, the user may be desired to maintain the action for a relatively long time to accurately measure the blood pressure.

### SUMMARY

Embodiments of the present disclosure provide a display device which reduces the time used to measure blood pressure and a method of measuring blood pressure using the display device.

Embodiments of the present disclosure also provide a display device which improves the accuracy of blood pressure measurement and a method of measuring blood pressure using the display device.

However, embodiments of the present disclosure are not restricted to the one set forth herein. The above and other features of embodiments of the present disclosure will become more apparent to one of ordinary skill in the art to which the present disclosure pertains by referencing the detailed description of the present disclosure given below:

According to an embodiment of the present disclosure, a display device comprises a first pixel which emits first light; a second pixel which emits second light having a different wavelength from the first light; a photosensor which senses the first light and the second light which are alternately provided a plurality of times; and a pulse wave sensing circuit which receives information about the first light and information about the second light sensed by the photosensor and generates a first pulse wave signal based on the information about the first light and a second pulse wave signal based on the information about the second light; and a main processor which receives the first pulse wave signal and the second pulse wave signal and calculates blood pressure information.

In an embodiment, the first pulse wave signal and the second pulse wave signal may be respectively generated based on the first light and the second light sensed in one full section, and the one full section may include a plurality of first unit sections comprising components of the first pulse wave signal, a plurality of second unit sections comprising components of the second pulse wave signal, and a plurality of transition points at which the first unit sections and the second unit sections are switched.

In an embodiment, the first pulse wave signal may include a plurality of first pulses located in the first unit sections, the second pulse wave signal comprises a plurality of second pulses located in the second unit sections, and the main processor is which generate, a first correction signal by inserting first virtual pulses, each of which is generated based on at least two first pulses adjacent to each other, into the second unit sections of the first pulse wave signal, and a second correction signal by inserting second virtual pulses, each of which is generated based on at least two second pulses adjacent to each other, into the first unit sections of the second pulse wave signal.

In an embodiment, each of the first virtual pulses may be generated through a linear interpolation using arbitrary points included in the at least two first pulses adjacent to each other, and each of the second virtual pulses may be generated through a linear interpolation using arbitrary points included in the at least two second pulses adjacent to each other.

In an embodiment, a first point of each of the first virtual pulses may be generated using a second point of a preceding pulse among the at least two first pulses adjacent to each other and a third point of a following pulse among the at least two first pulses adjacent to each other, coordinates of the first point are (xᵢ, yᵢ), coordinates of the second point are (xᵢ₋₁, yᵢ₋₁), coordinates of the third point are (xᵢ₊₁, yᵢ₊₁), and the first point of each of the first virtual pulses may satisfy the following equation:

$${y}_{i} = \frac{{y}_{i + 1} - {y}_{i - 1}}{{x}_{i + 1} - {x}_{i - 1}} ( {x}_{i} - {x}_{i - 1} ) + {y}_{i - 1} .$$

In an embodiment, each of the first unit sections may be a (2n−1)-th section in the full section, each of the second unit sections may be a (2n)-th section in the full section, and n is a natural number equal to or greater than 1.

In an embodiment, each of the first virtual pulses may be generated using an average value of the first pulses located in a (2i−3)-th section and a (2i−1)-th section among the first unit sections, each of the second virtual pulses may be generated using an average value of the second pulses located in a (2j−2)-th section and a (2j)-th section among the second unit sections, and i and j are natural numbers of 2 to n.

In an embodiment, the transition points may be lowest points of the first pulses and the second pulses, respectively.

In an embodiment, the one full section may end when an amplitude of a final pulse among the first pulses or an amplitude of a final pulse among the second pulses is smaller than a threshold value.

In an embodiment, the main processor may generate a third correction signal by removing noise from the second correction signal based on the first correction signal and the second correction signal, and the third correction signal may be generated based on a ratio of a first maximum value of the first correction signal and a second maximum value of the second correction signal.

In an embodiment, when the first correction signal is defined as CRS1, the first maximum value is defined as K1, the second correction signal is defined as CRS2, the second maximum value is defined as K2, and the third correction signal is defined as CRS3, wherein the third correction signal may be calculated by equation.

$$CRS 3 = CRS 2 - \frac{K 2}{K 1} CRS 1.$$

In an embodiment, the display device may further include a pressure sensor which senses a pressure applied from an outside, where the main processor may calculate blood pressure information based on a pressure measurement value measured by the pressure sensor and the third correction signal, generate a peak detection signal by connecting peak values of the third correction signal, calculate a first pressure measurement value corresponding to a maximum value of the peak detection signal, and calculate mean blood pressure equal to the first pressure measurement value, minimum blood pressure lower than the first pressure measurement value, and maximum blood pressure higher than the first pressure measurement value based on the first pressure measurement value.

In an embodiment, the minimum blood pressure may be defined as a minimum pressure measurement value among pressure measurement values corresponding to values corresponding to 60% to 80% of the maximum value of the peak detection signal, and the maximum blood pressure may be defined as a maximum pressure measurement value among the pressure measurement values corresponding to the values corresponding to 60% to 80% of the maximum value of the peak detection signal.

In an embodiment, the wavelength of the first light may be smaller than a wavelength of the second light.

In an embodiment, the first light may be green light or blue light, and the second light may be red light or infrared light.

In an embodiment, the display device may further include a display panel including the first pixel, the second pixel, and the photosensor, where the display panel may include a first light sensing area in which the first light is sensed and a second light sensing area in which the second light is sensed, and the first light sensing area is smaller in size than the second light sensing area in a plan view.

In an embodiment, the first and second light sensing areas may be surrounded by the first pixel and the second pixel, respectively, and a first light emitting area in which the first pixel emits light is larger in size than a second light emitting area in which the second pixel emits light.

According to an embodiment of the present disclosure, a method of measuring blood pressure using a display device which includes a first pixel which emits first light, a second pixel which emits second light having a different wavelength from the first light, a photosensor which senses the first light and the second light which are alternately provided a plurality of times, a pulse wave sensing circuit which receives information about the first light and information about the second light sensed by the photosensor and generates a first pulse wave signal based on the information about the first light and a second pulse wave signal based on the information about the second light, a pressure sensor which senses a pressure applied from an outside, and a main processor which receives the first pulse wave signal and the second pulse wave signal and calculates blood pressure information, the method including, measuring a pressure measurement value according to a pressure applying time through the pressure sensor, generating a correction signal based on the first pulse wave signal and the second pulse wave signal, and calculating blood pressure information based on the pressure measurement value and the correction signal.

In an embodiment, the generating the correction signal based on the first pulse wave signal and the second pulse wave signal may include generating first virtual pulses and second virtual pulses based on the first pulse wave signal and the second pulse wave signal, respectively, and generating a first correction signal and a second correction signal by inserting the first virtual pulses and the second virtual pulses into the first pulse wave signal and the second pulse wave signal, respectively.

In an embodiment, each of the first virtual pulses may be generated through a linear interpolation using arbitrary points included in at least two first pulses adjacent to each other among a plurality of first pulses of the first pulse wave signal, which is sensed in one full section, and each of the second virtual pulses may be generated through a linear interpolation using arbitrary points included in at least two second pulses adjacent to each other among a plurality of second pulses of the second pulse wave signal, which is sensed in the one full section.

A display device and a method of measuring blood pressure using the display device according to an embodiment may reduce the time used to measure blood pressure.

A display device and a method of measuring blood pressure using the display device according to an embodiment may improve the accuracy of blood pressure measurement.

### BRIEF DESCRIPTION OF THE DRAWINGS

These and/or other features of embodiments of the disclosure will become apparent and more readily appreciated from the following description of embodiments, taken in conjunction with the accompanying drawings, in which:

FIG. 1 is a schematic plan view of a display device according to an embodiment;

FIG. 2 is a plan view illustrating the configuration of the display device according to an embodiment:

FIG. 3 is a block diagram of the display device according to an embodiment:

FIG. 4 is a plan view illustrating pixels and photosensors of a display cell according to an embodiment:

FIG. 5 is a cross-sectional view taken along line I-I′ of FIG. 4;

FIG. 6 is a block diagram of a main processor according to an embodiment;

FIG. 7 is a block diagram illustrating an operation method when blood pressure is calculated according to an embodiment;

FIG. 8 is a plan view illustrating a light emitting area and a light sensing area of the display device according to an embodiment;

FIG. 9 is a plan view illustrating the light emitting area and the light sensing area when first light and second light are emitted and sensed;

FIG. 10 is a cross-sectional view taken along line II-II′ of FIG. 8;

FIG. 11 is a plan view illustrating a light emitting area and a light sensing area of a display device according to an embodiment;

FIG. 12 is a graph illustrating a first pulse wave signal and a second pulse wave signal according to an embodiment;

FIG. 13 is a graph illustrating a first correction signal and a second correction signal according to an embodiment;

FIG. 14 is a graph illustrating a process of generating the first correction signal and the second correction signal according to an embodiment;

FIGS. 15 through 17 are graphs illustrating a process of generating a third correction signal according to an embodiment:

FIG. 18 is a graph illustrating a process of calculating blood pressure information based on the third correction signal according to an embodiment:

FIG. 19 is a flowchart illustrating a method of measuring blood pressure using a display device according to an embodiment:

FIG. 20 is a graph illustrating a pressure measurement value in operation S100 according to an embodiment:

FIG. 21 is a flowchart illustrating operation S200 in FIG. 19 according to an embodiment:

FIG. 22 is a flowchart illustrating operation S300 in FIG. 19 according to an embodiment:

FIG. 23 is a flowchart illustrating operation S400 in FIG. 19 according to an embodiment:

FIG. 24 is a flowchart illustrating operation S500 in FIG. 19 according to an embodiment; and

FIG. 25 is a graph comparing measurement times in methods of measuring blood pressure using a conventional display device and a display device according to an embodiment.

### DETAILED DESCRIPTION

The invention will now be described more fully hereinafter with reference to the accompanying drawings, in which various embodiments of the invention are shown. This invention may, however, be embodied in different forms and should not be construed as limited to the embodiments set forth herein. Rather, these embodiments are provided so that this disclosure will be thorough and complete, and will filly convey the scope of the invention to those skilled in the art.

It will also be understood that when a layer is referred to as being “on” another layer or substrate, it can be directly on the other layer or substrate, or intervening layers may also be present therebetween. In contrast, when an element is referred to as being “directly on” another element, there are no intervening elements present. The same reference numbers indicate the same components throughout the specification.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a”, “an,” “the,” and “at least one” do not denote a limitation of quantity, and are intended to include both the singular and plural, unless the context clearly indicates otherwise. For example, “an element” has the same meaning as “at least one element,” unless the context clearly indicates otherwise. “At least one” is not to be construed as limiting “a” or “an.” “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

Hereinafter, embodiments of the disclosure will be described with reference to the accompanying drawings.

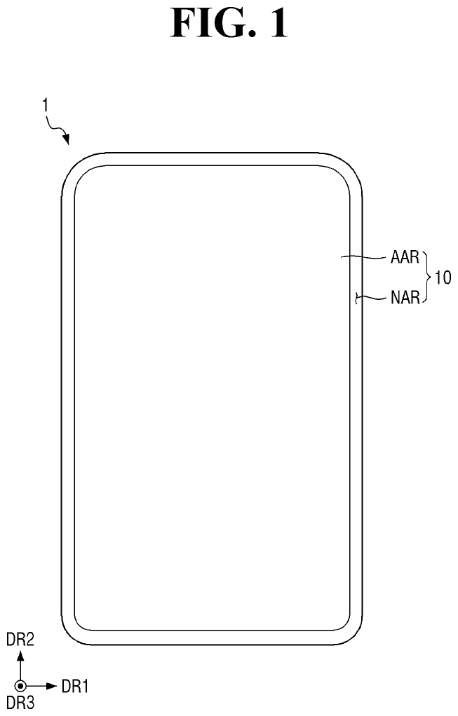

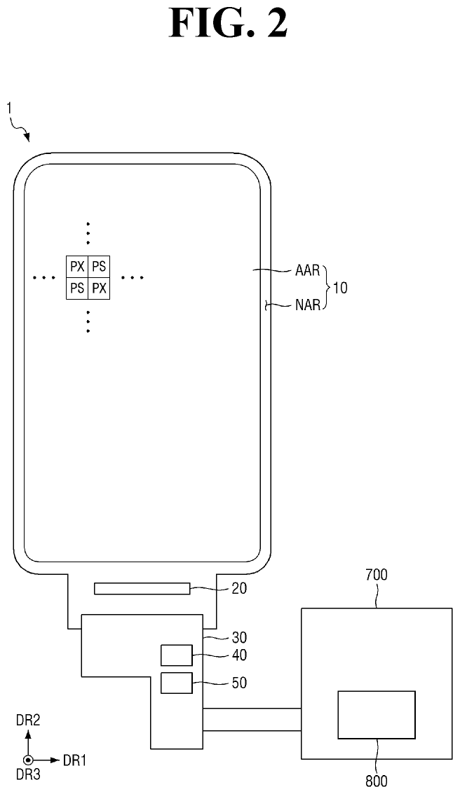

FIG. 1 is a schematic plan view of a display device 1 according to an embodiment. FIG. 2 is a plan view illustrating the configuration of the display device 1 according to an embodiment.

Referring to FIGS. 1 and 2, an embodiment of the display device 1 may refer to any electronic device that provides a display screen. Examples of the display device 1 may include, but not limited to, a mobile phone, a smartphone, a tablet personal computer (PC), a mobile communication terminal, an electronic notebook, an electronic book, a personal digital assistant (PDA), a portable multimedia player (PMP), a navigation device, an ultra-mobile PC (UMPC), a television, a game console, a wristwatch-type electronic device, a head mounted display, a monitor of a PC, a notebook computer, a car dashboard, a digital camera, a camcorder, an outdoor billboard, an electronic display board, various medical devices, various examination devices, various home appliances including a display area such as a refrigerator and a washing machine, and an Internet of things (IoT) device. A representative example of the display device 1 to be described below may be, but is not limited to, a smartphone, a tablet PC, or a notebook computer.

In an embodiment, as shown in FIG. 2, the display device 1 may include a display panel 10, a display driver 20, a circuit board 30, a pressure sensing circuit 40, a pulse wave sensing circuit 50, a main circuit board 700, and a main processor 800.

The display panel 10 may include an active area AAR and a non-active area NAR.

The active area AAR includes a display area in which an image is displayed. The active area AAR may completely overlap the display area. A plurality of pixels PX displaying an image may be disposed in the display area. Each pixel PX may include a light emitting unit that emits light.

The active area AAR further includes a light sensing area. The light sensing area is an area that reacts to light and an area configured to sense the amount or wavelength of incident light. A plurality of photo sensors PS that react to light may be disposed in the light sensing area. The light sensing area may overlap the display area. In an embodiment, the light sensing area may completely overlap the active area AAR in a plan view or when viewed in a thickness direction of the display panel 10, i.e., when viewed in a third direction DR3. In such an embodiment, the light sensing area and the display area may be the same as each other. In an embodiment, the light sensing area may be disposed only in a part of the active area AAR. In an embodiment, for example, the light sensing area may be disposed only in a limited area set for blood pressure measurement. In such an embodiment, the light sensing area may overlap a part of the display area but may not overlap the other part of the display area.

The non-active area NAR may be disposed around the active area AAR. The display driver 20 may be disposed in the non-active area NAR. The display driver 20 may drive the pixels PX and/or the photosensors PS. The display driver 20 may output signals and voltages for driving the display panel 10. The display driver 20 may be formed as an integrated circuit and mounted on the display panel 10. In the non-active area NAR, signal lines for transmitting signals between the display driver 20 and the active area AAR may be further disposed. In an embodiment, for example, the display driver 20 may be mounted on the circuit board 30.

The circuit board 30 may be attached to an end of the display panel 10 using an anisotropic conductive film (ACF). Lead lines of the circuit board 30 may be electrically connected to a pad portion of the display panel 10. The circuit board 30 may be a flexible printed circuit board or a flexible film such as a chip on film.

The pressure sensing circuit 40 may be disposed on the circuit board 30. The pressure sensing circuit 40 may be formed as (or defined by) an integrated circuit and attached to an upper surface of the circuit board 30. The pressure sensing circuit 40 may be connected to a display layer of the display panel 10. The pressure sensing circuit 40 may sense an electrical signal generated by pressure applied to a plurality of pressure sensors of the display panel 10. The pressure sensing circuit 40 may generate pressure data based on a change in the electrical signal sensed by the pressure sensors and transmit the pressure data to the main processor 800. The pressure sensing circuit 40 may form a pressure detection unit or a pressure sensor unit together with the pressure sensors.

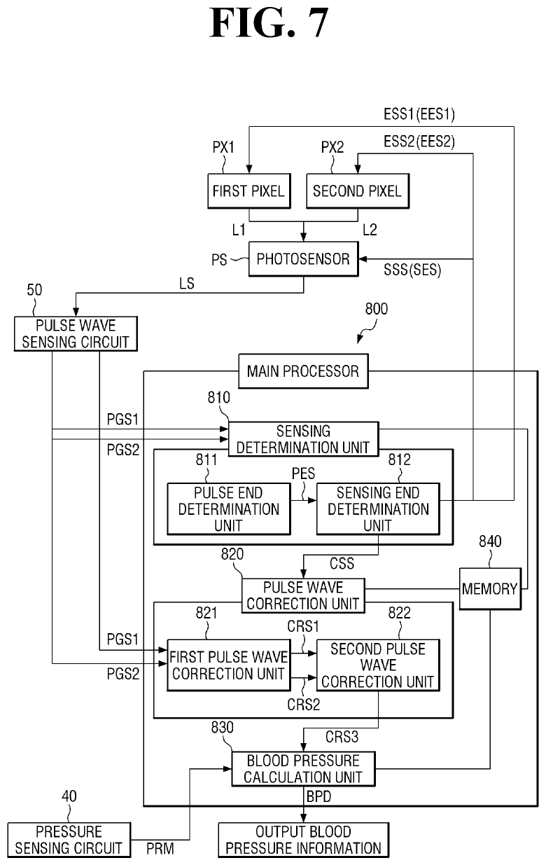

The pressure sensing circuit 40 may measure and calculate the pressure applied by a user and sensed through the pressure sensors as a pressure measurement value PRM (shown in FIG. 7). When a user applies a gradually increasing pressure in the process of bringing his or her finger into contact with the display device 1, the diameter of blood vessels may be reduced, causing blood flow to decrease or become substantially zero. By tracking this change in blood volume based on the change in pressure, it is possible to calculate blood pressure information. The change in blood volume may be measured in the form of a first pulse wave signal PGS1 and a second pulse wave signal PGS2 through the pulse wave sensing circuit 50.

The pulse wave sensing circuit 50 may be disposed on the circuit board 30. The pulse wave sensing circuit 50 may be formed as an integrated circuit and attached to the upper surface of the circuit board 30. The pulse wave sensing circuit 50 may be connected to the display layer of the display panel 10. The pulse wave sensing circuit 50 may sense a photocurrent (e.g., photo information) generated by photocharges incident on the photosensors PS of the display panel 10. The pulse wave sensing circuit 50 may recognize a user's pulse wave based on the photocurrent.

During the systole of the heart, the blood ejected from the left ventricle of the heart moves to peripheral tissues, thus increasing blood volume on the arterial side. In addition, during the systole of the heart, red blood cells carry more oxy hemoglobin to the peripheral tissues. During the diastole of the heart, there is partial suction of blood from the peripheral tissues toward the heart. Here, when light emitted from the pixels PX is radiated to arteries, the radiated light may be absorbed by the peripheral tissues such as skin and blood vessels. Light absorbance is dependent on hematocrit and blood volume. The light absorbance may have a maximum value during the systole of the heart and a minimum value during the diastole of the heart. Since the light absorbance is inversely proportional to the amount of light incident on each photosensor PS, it is possible to estimate the light absorbance at a corresponding time through light reception data regarding the amount of light incident on each photosensor PS.

The amount of light incident on the photosensors PS may include not only heartbeat data but also other noises. In an embodiment, for example, the heartbeat data may be generated when the photosensors PS receive light reflected from arteries inside the skin tissue. The noises may be generated when the photosensors PS receive light reflected from capillaries, tissues, and skin.

Pulse wave signals generated by the pulse wave sensing circuit 50 may include a pulse wave signal corresponding to noise as well as a pulse wave signal corresponding to heartbeat. The display device 1 according to an embodiment may sense light of various wavelengths and generate a pulse wave signal from which noise has been removed by using various pulse wave signals generated by the pulse wave sensing circuit 50 and may calculate accurate blood pressure information based on the pulse wave signal.

The main circuit board 700 may be a printed circuit board or a flexible printed circuit board. The main circuit board 700 may be electrically connected to the circuit board 30 through a connection film or the like.

The main circuit board 700 may include the main processor 800.

The main processor 800 may control all functions of the display device 1. In an embodiment, for example, the main processor 800 may output digital video data to the display driver 20 through the circuit board 30 so that the display panel 10 displays an image. In addition, the main processor 800 may receive touch data from a touch driving circuit (not illustrated), determine coordinates of a user's touch, and then execute an application indicated by an icon displayed at the coordinates of the user's touch.

The main processor 800 may be an application processor formed as an integrated circuit, a central processing unit, or a system chip.

The main processor 800 may calculate a pulse wave signal that reflects a blood change corresponding to heartbeat based on an optical signal received from the pulse wave sensing circuit 50. In addition, the main processor 800 may calculate a user's touch pressure (e.g., a pressure measurement value) based on an electrical signal received from the pressure sensing circuit 40. Then, the main processor 800 may calculate the user's blood pressure based on the pulse wave signal and the pressure signal.

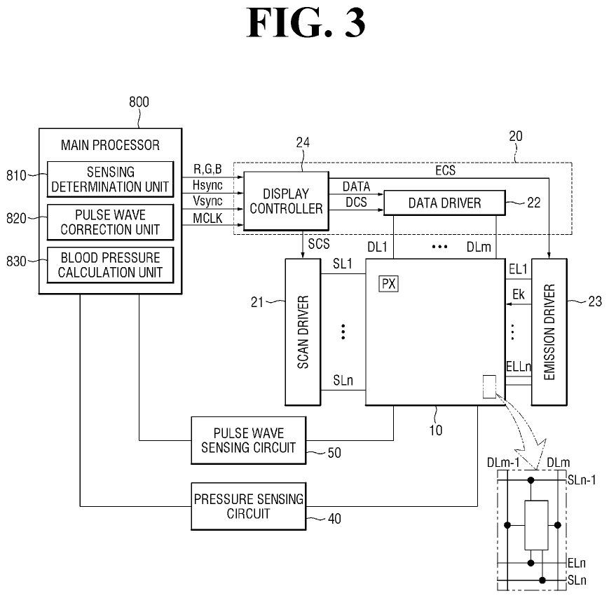

The main processor 800 may generate a plurality of pulse wave signals PGS1 and PGS2 (see FIG. 7) alternately sensed from light having different wavelengths through a sensing determination unit 810 (see FIG. 3) which will be described later and may generate correction signals CRS1 and CRS2 (see FIG. 7) reconstructed from the pulse wave signals through a pulse wave correction unit 820 (see FIG. 3) which will be described later. This will be described later in greater detail.

In the display device 1 according to an embodiment, the main processor 800 may correct pulse wave data measured by the pulse wave sensing circuit 50 and calculate blood pressure information by comparing the pulse wave data with pressure data measured by the pressure sensing circuit 40.

FIG. 3 is a block diagram of the display device 1 according to an embodiment.

Referring to FIG. 3, an embodiment of the display device 1 may include the display panel 10 including a plurality of pixels PX, the display driver 20, a scan driver 21, an emission driver 23, the pressure sensing circuit 40, the pulse wave sensing circuit 50, and the main processor 800.

The main processor 800 may drive and control a display controller 24, the pressure sensing circuit 40, and the pulse wave sensing circuit 50. The main processor 800 may include the sensing determination unit 810, the pulse wave correcting unit 820, and a blood pressure calculation unit 830.

The sensing determination unit 810 may receive an optical signal from the pulse wave sensing circuit 50 and determine whether a pulse of a pulse wave signal has ended and whether sensing has ended.

The pulse wave correction unit 820 may generate a correction signal by reconstructing a pulse wave signal. The pulse wave signal may not include pulses in some sections, and the pulse wave correction unit 820 may generate a correction signal by inserting virtual pulses into the sections not including pulses.

The blood pressure calculation unit 830 may calculate blood pressure information based on pressure data received from the pressure sensing circuit 40 and corrected pulse wave data received from the pulse wave correction unit 820.

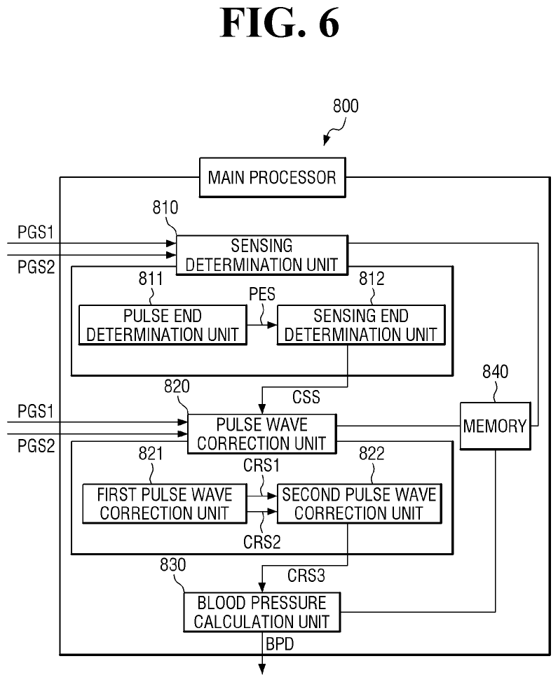

The sensing determination unit 810, the pulse wave correction unit 820, and the blood pressure calculation unit 830 of the main processor 800 will be described later with reference to FIG. 6, etc.

The main processor 800 may output image information to the display controller 24. In an embodiment, for example, the main processor 800 may output image information including the calculated pulse wave signal, blood pressure measurement value, and blood pressure information to the display controller 24.

The display controller 24 may convert a plurality of image signals R, G, B supplied from the main processor 800 into a plurality of image data DATA and send the image data DATA to a data driver 22 together with a data control signal DCS. In addition, the display controller 24 may receive a vertical synchronization signal Vsync, a horizontal synchronization signal Hsync and a clock signal MCLK from the main processor 800, generate control signals for controlling the driving of the scan driver 21, the data driver 22 and the emission driver 23 based on the vertical synchronization signal Vsync, the horizontal synchronization signal Hsync and the clock signal MCLK, and transmit the control signals to the scan driver 21, the data driver 22 and the emission driver 23, respectively. In an embodiment, for example, the display controller 24 may generate a scan control signal SCS for controlling the operation timing of the scan driver 21, an emission control signal ECS for controlling the operation timing of the emission driver 23, and the data control signal DCS for controlling the operation timing of the data driver 22. The display controller 24 may output the scan control signal SCS to the scan driver 21 and output the emission control signal ECS to the emission driver 23.

The display controller 24 may be electrically connected to the display panel 10 and/or the main processor 800 through wiring or through a communication network. In an embodiment, at least a portion of the display controller 24 may be directly attached onto the display panel 10 in the form of a driving chip.

The data driver 22 may receive the image data DATA and the data control signal DCS from the display controller 24. The data driver 22 may convert the image data DATA into analog data voltages based on the data control signal DCS. The data driver 22 may output the analog data voltages to data lines DL1 through DLm in synchronization with a scan signal.

The scan driver 21 may generate scan signals in response to the scan control signal SCS and sequentially output the scan signals to scan lines SL1 through SLn.

Although not illustrated in the drawing, a driving voltage line (not illustrated), a common voltage line (not illustrated), and a power voltage line (not illustrated) may be further included. The power voltage line may include the driving voltage line and the common voltage line. A driving voltage may be a high potential voltage for driving a light emitting element and a photoelectric converter, and a common voltage may be a low potential voltage for driving the light emitting element and the photoelectric converter. That is, the driving voltage may have a higher potential than the common voltage.

Display control signals may include the scan control signal SCS, the data control signal DCS, and the emission control signal ECS. The display control signals may be output from the display controller 24 and provided to the scan driver 21, the data driver 22, and the emission driver 23, respectively.

The emission driver 23 may generate emission signals Ek in response to the emission control signal ECS and sequentially output the emission signals Ek to emission lines ELL1 through ELLn. In an embodiment, as shown in FIG. 3, the emission driver 23 may be provided separately from the scan driver 21, the present disclosure is not limited thereto, and alternatively, the emission driver 23 may also be included in the scan driver 21.

The data driver 22 and the display controller 24 may be included in the display driver 20 that controls the operation of the display panel 10. The data driver 22 and the display controller 24 may be formed as integrated circuits and mounted on the display driver 20.

Each of the pixels PX may be connected to a corresponding one of the scan lines SL1 through SLn, a corresponding one of the data lines DL1 through DLm, and a corresponding one of the emission lines ELL1 through ELLn.

Each of the photosensors PS may be connected to a corresponding one of the scan lines SL1 through SLn and a corresponding one of readout lines (not illustrated).

The scan lines SL1 through SLn may connect the scan driver 21 to the pixels PX and the photosensors PS. The scan lines SL1 through SLn may provide scan signals output from the scan driver 21 to the pixels PX.

The data lines DL1 through DLm may connect the data driver 22 to the pixels PX. The data lines DL1 through DLm may provide image data output from the data driver 22 to the pixels PX.

The emission lines ELL1 through ELLn may connect the emission driver 23 to the pixels PX. The emission lines ELL1 through ELLn may provide emission signals EK output from the emission driver 23 to the pixels PX.

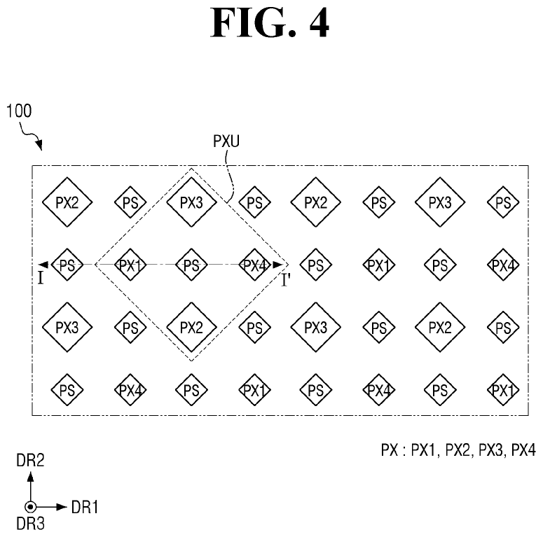

FIG. 4 is a plan view illustrating pixels PX and photosensors PS of a display cell 100 according to an embodiment.

Referring to FIG. 4, a plurality of pixels PX and a plurality of photosensors PS may be repeatedly disposed in the display cell 100. A plurality of display cells 100 may be defined or formed in the display panel 10.

The pixels PX may include first pixels PX1, second pixels PX2, third pixels PX3, and fourth pixels PX4. In an embodiment, for example, the second pixels PX2 may emit light of a red wavelength, the first pixels PX1 and the fourth pixels PX4 may emit light of a green wavelength, and the third pixels PX3 may emit light of a blue wavelength.

However, the present disclosure is not limited thereto. As will be described later, in an embodiment, when blood pressure is measured, the first pixels PX1 may emit green light, and the second pixels PX2 may emit infrared light. Alternatively, the first pixels PX1 may emit blue light or green light, and the second pixels PX2 may emit red light or infrared light. For ease of description, embodiments where the first pixels PX1 emit green light and the second pixels PX2 emit red light will be described below as an example.

When blood pressure is measured, the shorter the wavelength of light emitted from the pixels PX, the smaller the depth to which the light can penetrate the human body. In an embodiment, for example, blue light can reach near the epidermis, green light can reach capillaries, and red light and infrared light can reach arteries. Therefore, signals obtained from the red light and the infrared light may include changes caused not only by the arteries but also by the capillaries. In general, blood pressure is measured by the pressure applied to blood vessel walls of the arteries. Thus, changes caused by the capillaries may act as noise when blood pressure is measured. A method of removing such noise will be described later.

The pixels PX may respectively include a plurality of light emitting areas that emit light. The photosensors PS may include a plurality of light sensing areas that sense incident light.

The first pixels PX1, the second pixels PX2, the third pixels PX3, the fourth pixels PX4, and the photosensors PS may be alternately arranged in a first direction DR1 and a second direction DR2. In an embodiment, the second pixels PX2 and the third pixels PX3 may be alternately arranged along the first direction DR1 to form a first row, and the first pixels PX1 and the fourth pixels PX4 may be alternately arranged along the first direction DR1 in a second row adjacent to the first row. The pixels PX in the first row may be staggered with the pixels PX in the second row in the first direction DR1. The arrangement of the first row and the second row may be repeated up to an n^{th} row.

In the drawing, the first direction DR1 and the second direction DR2 intersect each other as horizontal directions. For example, the first direction DR1 and the second direction DR2 may be orthogonal to each other. In addition, the third direction DR3 may intersect the first direction DR1 and the second direction DR2 and may be, for example, a vertical direction orthogonal to the first direction DR1 and the second direction DR2. In the present specification, a direction indicated by an arrow of each of the first through third directions DR1 through DR3 may be referred to as one side, and the opposite direction may be referred to as the other side.

A plurality of photosensors PS may be respectively disposed between the second pixels PX2 and the third pixels PX3 forming the first row. The second pixels PX2, the photosensors PS, and the third pixels PX3 may be alternately arranged along the first direction DR1. A plurality of photosensors PS may be respectively disposed between the first pixels PX1 and the fourth pixels PX4 forming the second row. The first pixels PX1, the photosensors PS, and the fourth pixels PX4 may be alternately arranged along the first direction DR1. The number of photosensors PS in the first row may be equal to the number of photosensors PS in the second row. The arrangement of the first row and the second row may be repeated up to the n^{th} row.

In an alternative embodiment, for example, the photosensors PS may be disposed between the first pixels PX1 and the fourth pixels PX4 forming the second row but may not be disposed between the second pixels PX2 and the third pixels PX3 forming the first row. That is, the photosensors PS may not be disposed in the first row.

The light emitting areas of the pixels PX may have different sizes (e.g., planar areas) from each other. The light emitting areas of the first and fourth pixels PX1 and PX4 may be smaller than the light emitting areas of the second and third pixels PX2 and PX3. In an embodiment, as shown in FIG. 4, each pixel PX may have a rhombus shape, but the present disclosure is not limited thereto, and alternatively, each pixel PX may also have a rectangular, octagonal, circular, or other polygonal shape.

One pixel unit PXU may include (or be defined by) one first pixel PX1, one second pixel PX2, one third pixel PX3, and one fourth pixel PX4. The pixel unit PXU refers to a group of color pixels that can express a gray level.

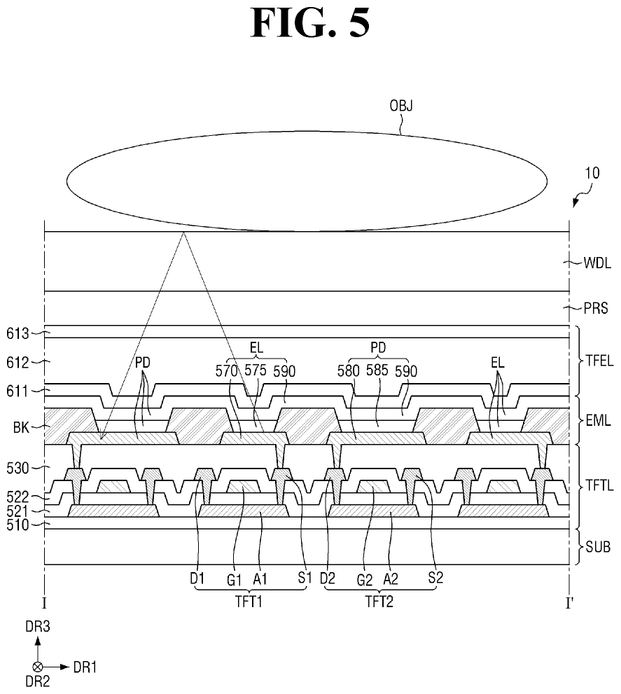

FIG. 5 is a cross-sectional view taken along line I-I′ of FIG. 4.

Referring to FIG. 5, an embodiment of the display panel 10 may include a substrate SUB, a thin-film transistor layer TFTL, a light emitting element layer EML, a thin-film encapsulation layer TFEL, a pressure sensing layer PRS, and a window layer WDL.

The substrate SUB may be a flexible substrate that can be bent, folded, or rolled. In an embodiment, for example, the substrate SUB may include polymer resin such as polyimide (PI), but the present disclosure is not limited thereto. In an alternative embodiment, the substrate SUB may be a rigid substrate including a glass material or a metal material.

The thin-film transistor layer TFTL may be disposed on the substrate SUB. The thin-film transistor layer TFTL may include a buffer layer 510, a first thin-film transistor TFT1, a second thin-film transistor TFT2, a gate insulating layer 521, an interlayer insulating layer 522, and a planarization layer 530.

The buffer layer 510 may include an inorganic layer that can prevent penetration of air or moisture. In an embodiment, for example, the buffer layer 510 may include silicon nitride, silicon oxide, or silicon oxynitride.

The first thin-film transistor TFT1 and the second thin-film transistor TFT2 may be disposed on the buffer layer 510. The thin-film transistors TFT1 and TFT2 may respectively include semiconductor layers A1 and A2, the gate insulating layer 521 disposed on portions of the semiconductor layers A1 and A2, gate electrodes G1 and G2 on the gate insulating layer 521, the interlayer insulating layer 522 covering the semiconductor layers A1 and A2 and the gate electrodes G1 and G2, and source electrodes S1 and S2 and drain electrodes D1 and D2 on the interlayer insulating layer 522.

The semiconductor layers A1 and A2 may form channels of the first thin-film transistor TFT1 and the second thin-film transistor TFT2, respectively. The semiconductor layers A1 and A2 may include polycrystalline silicon. In an embodiment, the semiconductor layers A1 and A2 may include monocrystalline silicon, low-temperature polycrystalline silicon, amorphous silicon, or an oxide semiconductor. The oxide semiconductor may include, for example, a binary compound (ABx), a ternary compound (ABxCy) or a quaternary compound (ABxCyDz) containing indium, zinc, gallium, tin, titanium, aluminum, hafnium (Hf), zirconium (Zr), magnesium (Mg), etc. Each of the semiconductor layers A1 and A2 may include a channel region, and source and drain regions doped with impurities.

The gate insulating layer 521 may be disposed on the semiconductor layers A1 and A2. The gate insulating layer 521 may electrically insulate a first gate electrode G1 from a first semiconductor layer A1 and electrically insulate a second gate electrode G2 from a second semiconductor layer A2. The gate insulating layer 521 may include or be made of an insulating material such as silicon oxide (SiOₓ), silicon nitride (SiNₓ), or metal oxide.

The first gate electrode G1 of the first thin-film transistor TFT1 and the second gate electrode G2 of the second thin-film transistor TFT2 may be disposed on the gate insulating layer 521. The gate electrodes G1 and G2 may be respectively disposed above the channel regions of the semiconductor layers A1 and A2, that is, may be disposed on the gate insulating layer 521 at positions overlapping the channel regions.

The interlayer insulating layer 522 may be disposed on the gate electrodes G1 and G2. The interlayer insulating layer 522 may include an inorganic insulating material such as silicon oxide (SiOₓ), silicon nitride (SiNₓ), silicon oxynitride, hafnium oxide, or aluminum oxide. Although not illustrated, the interlayer insulating layer 522 may include a plurality of insulating layers and may further include a conductive layer between the insulating layers to form a capacitor second electrode.

The source electrodes S1 and S2 and the drain electrodes D1 and D2 may be disposed on the interlayer insulating layer 522. A first source electrode S1 of the first thin-film transistor TFT1 may be electrically connected to the drain region of the first semiconductor layer A1 through a contact hole defined through the interlayer insulating layer 522 and the gate insulating layer 521. A second source electrode S2 of the second thin-film transistor TFT2 may be electrically connected to the drain region of the second semiconductor layer A2 through a contact hole defined through the interlayer insulating layer 522 and the gate insulating layer 521. Each of the source electrodes S1 and S2 and the drain electrodes D1 and D2 may include at least one metal selected from aluminum (Al), molybdenum (Mo), platinum (Pt), palladium (Pd), silver (Ag), magnesium (Mg), gold (Au), nickel (Ni), neodymium (Nd), iridium (Ir), chromium (Cr), calcium (Ca), titanium (Ti), tantalum (Ta), tungsten (W), and copper (Cu).

The planarization layer 530 may be disposed on the interlayer insulating layer 522 to cover the source electrodes S1 and S2 and the drain electrodes D1 and D2. The planarization layer 530 may include or be made of an organic insulating material or the like. The planarization layer 530 may have a flat surface and may include contact holes exposing one of the source electrodes S1 and S2 and one of the drain electrodes D1 and D2.

The light emitting element layer EML may be disposed on the planarization layer 530. The light emitting element layer EML may include light emitting elements EL, photoelectric converters PD, and a bank layer BK. Each of the light emitting elements EL may include a pixel electrode 570, a light emitting layer 575, and a common electrode 590. Each of the photoelectric converters PD may include a first electrode 580, a photoelectric conversion layer 585, and the common electrode 590.

The pixel electrode 570 of each light emitting element EL may be disposed on the planarization layer 530. The pixel electrode 570 may be provided for each pixel PX. The pixel electrode 570 may be connected to the first source electrode S1 or first drain electrode D1 of the first thin-film transistor TFT1 through a contact hole penetrating the planarization layer 530.

The pixel electrode 570 of each light emitting element EL may have, but not limited to, a single layer structure of molybdenum (Mo), titanium (Ti), copper (Cu) or aluminum (Al) or a laminated layer structure, for example, a multilayer structure of ITO/Mg, ITO/MgF, ITO/Ag or ITO/Ag/ITO including indium tin oxide (ITO), indium zinc oxide (IZO), zinc oxide (ZnO) or indium oxide (In₂O₃) and silver (Ag), magnesium (Mg), aluminum (Al), platinum (Pt), lead (Pd), gold (Au) or nickel (Ni).

The first electrode 580 of each photoelectric converter PD may also be disposed on the planarization layer 530. The first electrode 580 of each photoelectric converter PD may be disposed in (or directly on) a same layer as the pixel electrode 570 of each light emitting element EL. The first electrode 580 of each photoelectric converter PD may be disposed on one side of the pixel electrode 570 of each light emitting element EL. The first electrode 580 may be provided for each photosensor PS. The first electrode 580 may be connected to the second source electrode S2 or second drain electrode D2 of the second thin-film transistor TFT2 through a contact hole defined through the planarization layer 530.

The first electrode 580 of each photoelectric converter PD may have, but not limited to, a single layer structure of molybdenum (Mo), titanium (Ti), copper (Cu) or aluminum (Al) or a multilayer structure of ITO/Mg, ITO/MgF, ITO/Ag or ITO/Ag/ITO.

The bank layer BK may be disposed on the pixel electrode 570 and the first electrode 580. The bank layer BK may be provided with an opening defined or formed therein at each area overlapping the pixel electrode 570 to expose the pixel electrode 570. The opening exposing the pixel electrode 570 may provide a space in which the light emitting layer 575 of each light emitting element EL is disposed or formed, and an area in which the exposed pixel electrode 570 and the light emitting layer 575 overlap each other may be defined as a light emitting area that emits different light according to each pixel PX.

The bank layer BK may be provided with an opening defined or formed therein at each area overlapping the first electrode 580 to expose the first electrode 580. The opening exposing the first electrode 580 may provide a space in which the photoelectric conversion layer 585 of each photosensor PS is disposed or formed, and an area in which the exposed first electrode 580 and the photoelectric conversion layer 585 overlap each other may be defined as a light sensing area.

The bank layer BK may include an organic insulating material such as polyacrylates resin, epoxy resin, phenolic resin, polyamides resin, polyimides resin, unsaturated polyesters resin, polyphenylenethers resin, polyphenylenesulfides resin, or benzocyclobutene (BCB). In an embodiment, for example, the bank layer BK may include an inorganic material such as silicon nitride.

The light emitting layer 575 may be disposed on the pixel electrode 570 of each light emitting element EL exposed by an opening of the bank layer BK. The light emitting layer 575 may include a high molecular material or a low molecular material and may emit red, green, blue or infrared light in each pixel PX. Light emitted from the light emitting layer 575 may contribute to image display or may function as a light source incident on a photosensor PS. In an embodiment, for example, a green wavelength light emitted from the light emitting areas of the first and fourth pixels PX1 and PX4 may function as a light incident on the light sensing areas of the photosensors PS. A red wavelength light emitted from the second pixels PX2 and a blue wavelength light emitted from the third pixels PX3 may also perform a similar function.

The photoelectric conversion layer 585 may be disposed on the first electrode 580 of each photoelectric converter PD exposed by an opening of the bank layer BK. An area where the exposed first electrode 580 and the photoelectric conversion layer 585 overlap may be defined as a light sensing area of each photosensor PS. The photoelectric conversion layer 585 may generate photocharges in proportion to incident light. The incident light may be light entering the photoelectric conversion layer 585 after being emitted from the light emitting layer 575 and then reflected or may be light provided from the outside regardless of the light emitting layer 575. Charges generated and accumulated in the photoelectric conversion layer 585 may be converted into electrical signals for sensing.

The photoelectric conversion layer 585 may include an electron donor material and an electron acceptor material. The electron donor material may generate donor ions in response to light, and the electron acceptor material may generate acceptor ions in response to light. In an embodiment where the photoelectric conversion layer 585 includes or is made of an organic material, the electron donor material may include, but not limited to, a compound such as subphthalocyanine (SubPc) or dibutylphosphate (DBP). The electron acceptor material may include, but not limited to, a compound such as fullerene, a fullerene derivative, or perylene diimide.

In an embodiment, where the photoelectric conversion layer 585 includes or is made of an inorganic material, each photoelectric converter PD may be a pn-type or pin-type phototransistor. In an embodiment, for example, the photoelectric conversion layer 585 may have a structure in which an N-type semiconductor layer, an I-type semiconductor layer, and a P-type semiconductor layer are sequentially stacked.

In an embodiment, where the photoelectric conversion layer 585 includes or is made of an organic material, a hole injecting layer (HIL) and a hole transporting layer (HTL) may be disposed under the photoelectric conversion layer 585, and an electron injecting layer (EIL) and an electron transporting layer (ETL) may be stacked on the photoelectric conversion layer 585. Each of the above layers may be a single layer or a multilayer including one or more organic materials.

The common electrode 590 may be disposed on the light emitting layer 575, the photoelectric conversion layer 585, and the bank layer BK. The common electrode 590 may be disposed over a plurality of pixels PX and a plurality of photosensors PS to cover the light emitting layers 575, the photoelectric conversion layers 585, and the bank layer BK. The common electrode 590 may include a conductive material having a low work function, such as Li, Ca, LiF/Ca, LiF/Al, Al, Mg, Ag, Pt, Pd, Ni, Au Nd, Ir, Cr, BaF, Ba, or a compound or mixture thereof (e.g., a mixture of Ag and Mg). Alternatively, the common electrode 590 may include a transparent metal oxide such as indium tin oxide (ITO), indium zinc oxide (IZO), or zinc oxide (ZnO).

The common electrode 590 may be commonly disposed on the light emitting layers 575 and the photoelectric conversion layers 585, although the present disclosure is not limited thereto. In an embodiment, cathodes of the light emitting elements EL and sensing cathodes of the photoelectric converters PD may be electrically connected to each other. In an embodiment, for example, a common voltage line connected to the cathodes of the light emitting elements EL may be simultaneously connected to the sensing cathodes of the photoelectric converters PD.

The thin-film encapsulation layer TFEL may be disposed on the light emitting element layer EML. The thin-film encapsulation layer TFEL may include at least one inorganic layer to prevent penetration of oxygen or moisture into the light emitting layers 575 and the photoelectric conversion layers 585. In addition, the thin-film encapsulation layer TFEL may include at least one organic layer to protect the light emitting layers 575 and the photoelectric conversion layers 585 from foreign substances such as dust. In an embodiment, for example, the thin-film encapsulation layer TFEL may be formed in a structure in which a first inorganic layer 611, an organic layer 612, and a second inorganic layer 613 are sequentially stacked. Each of the first inorganic layer 611 and the second inorganic layer 613 may be formed as a multilayer in which one or more inorganic layers, each including at least one selected from a silicon nitride layer, a silicon oxynitride layer, a silicon oxide layer, a titanium oxide layer, and an aluminum oxide layer, are alternately stacked. The organic layer 612 may be an organic layer such as acryl resin, epoxy resin, phenolic resin, polyamide resin, or polyimide resin.

The pressure sensing layer PRS may be disposed on the thin-film encapsulation layer TFEL. The pressure sensing layer PRS may be provided in the form of a panel or a film and may be attached onto the thin-film encapsulation layer TFEL through a bonding layer such as PSA. Since the pressure sensing layer PRS is located on a light emission path of each light emitting layer 575, it may have high transmittance.

The pressure sensing layer PRS may sense pressure applied to the display device 1. When a user touches an upper surface of the display device 1, the pressure of the touch input may be sensed by the pressure sensing layer PRS. In an embodiment, a pressure sensing electrode of the pressure sensing layer PRS may be formed directly on a touch layer (not illustrated) disposed between the thin-film encapsulation layer TFEL and the pressure sensing layer PRS. In such an embodiment, the pressure sensing layer PRS may be internalized in the display panel 10 together with the touch layer (not illustrated).

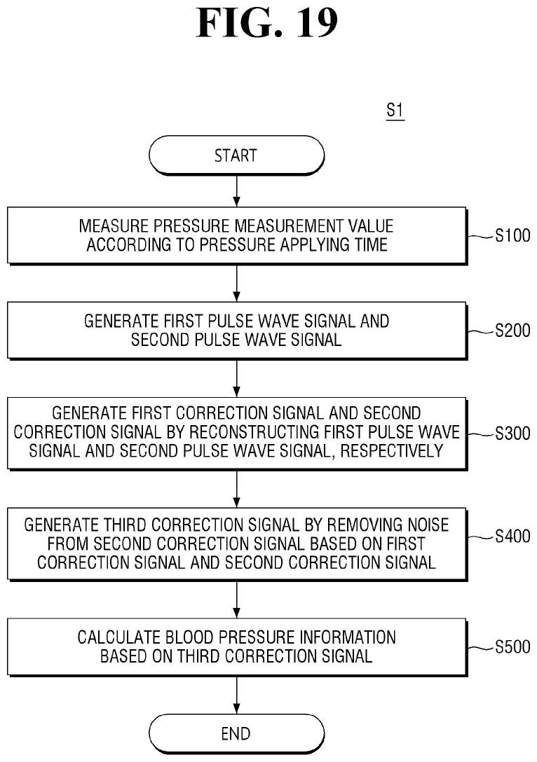

The pressure sensing layer PRS may be used to measure a pressure measurement value used to calculate blood pressure information in a method S1 (see FIG. 19) of measuring blood pressure using a display device, which will be described later. The pressure sensing layer PRS may sense the pressure applied by a user to the upper surface of the display device 1 and transmit the sensed pressure to the pressure sensing circuit 40.

The window layer WDL may be disposed on the pressure sensing layer PRS. The window layer WDL may be disposed on the display device 1 after a cutting process and a module process are performed on the display cell 100 and thus may protect the elements of the display device 1. The window layer WDL may include a glass or plastic material.

FIG. 5 is a cross-sectional view showing a user's finger OBJ in contact with the window layer WDL of the display device 1. When the user's finger OBJ touches an upper surface of the window layer WDL, light output from the light emitting areas of the pixels PX may be reflected by the user's finger OBJ. In this case, the blood flow in the blood vessels of the user's finger OBJ may vary according to the pressure applied to the user's finger OBJ. The blood flow in the blood vessels of the user's finger OBJ may be derived based on a difference in the amount of light reflected by the user's finger OBJ and then incident on the photosensors PS. In the display device 1 according to an embodiment, the user's blood pressure can be measured through the photosensors PS and the pressure sensing layer PRS.

FIG. 6 is a block diagram of a main processor 800 according to an embodiment.

Referring to FIG. 6, an embodiment of the main processor 800 may include a sensing determination unit 810, a pulse wave correction unit 820, a blood pressure calculation unit 830, and a memory 840.

The sensing determination unit 810 may receive an optical signal from the pulse wave sensing circuit 50. In an embodiment, for example, the sensing determination unit 810 may receive a first pulse wave signal PGS1 and a second pulse wave signal PGS2 from the pulse wave sensing circuit 50. The sensing determination unit 810 may determine whether the first pulse wave signal PGS1 and the second pulse wave signal PGS2 have ended and whether sensing has ended (or is completed). In an embodiment, for example, the sensing determination unit 810 may include a pulse end determination unit 811 and a sensing end determination unit 812.

The pulse wave correction unit 820 may receive a correction start signal CSS from the sensing determination unit 810. The pulse wave correction unit 820 may generate a first correction signal CRS1 and a second correction signal CRS2 by reconstructing the first pulse wave signal PGS1 and the second pulse wave signal PGS2, respectively, and may generate a third correction signal CRS3 based on the first correction signal CRS1 and the second correction signal CRS2. In an embodiment, for example, the pulse wave correction unit 820 may include a first pulse wave correction unit 821 and a second pulse wave correction unit 822.

In some embodiments, each of the first pulse wave signal PGS1 and the second pulse wave signal PGS2 may not include pulses in some sections, and the pulse wave correction unit 820 may generate the first correction signal CRS1 and the second correction signal CRS2 by inserting virtual pulses into the sections not including pulses.

In some embodiments, the first correction signal CRS1 generated from the first pulse wave signal PGS1 may include blood changes caused by capillaries as information, and the second correction signal CRS2 generated from the second pulse wave signal PGS2 may include blood changes caused by capillaries and arteries as information. The pulse wave correction unit 820 may generate the third correction signal CRS3 based on the first correction signal CRS1 and the second correction signal CRS2 to extract only the blood changes caused by the arteries.

The blood pressure calculation unit 830 may calculate blood pressure information BPD based on the pressure measurement value PRM (shown in FIG. 7) received from the pressure sensing circuit 40 and the third correction signal CRS3 received from the pulse wave correction unit 820.

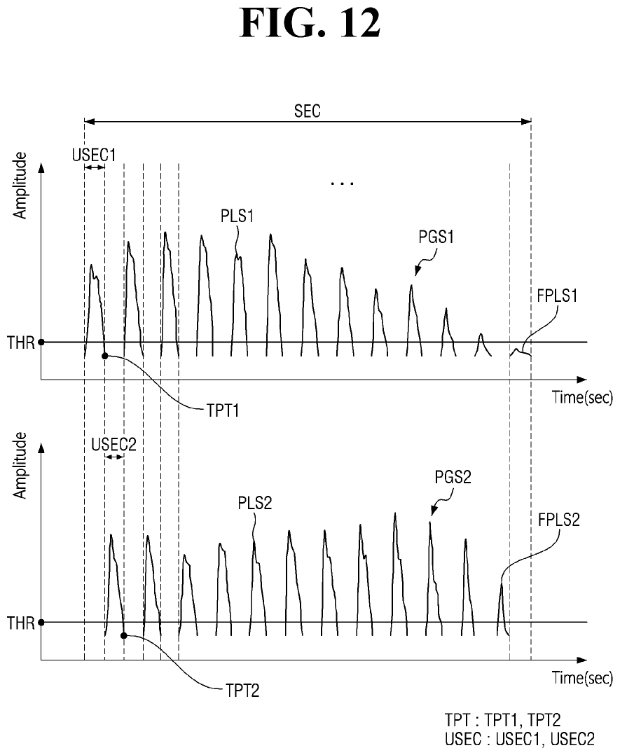

The memory 840 may store information used for the operation processing of the sensing determination unit 810, the pulse wave correction unit 820, and the blood pressure calculation unit 830. In an embodiment, for example, the memory 840 may store a threshold value THR (see FIG. 12) used for the sensing determination unit 810 to determine whether sensing has ended. In an alternative embodiment, for example, the memory 840 may store information used for the pulse wave correction unit 820 to remove noise for each user, that is, data used to remove noise based on the amount of light reflected from a user's capillaries, skin, tissues, etc.

An operation method of each element of the main processor 800 when blood pressure is calculated will be described below with reference to FIG. 7, etc.

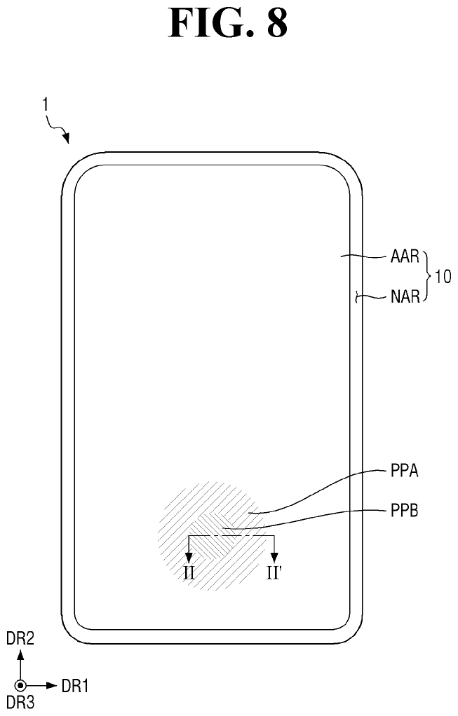

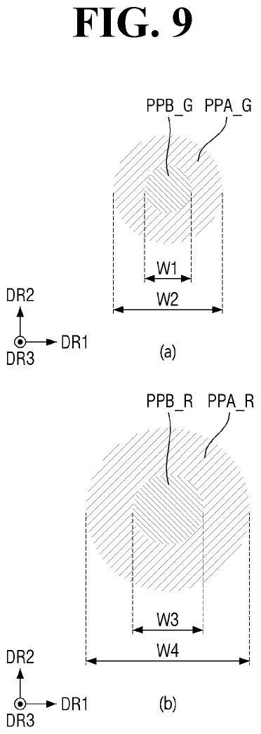

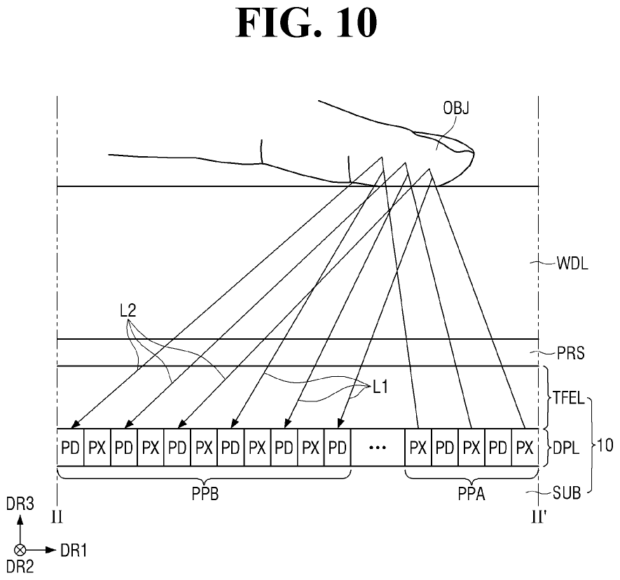

FIG. 7 is a block diagram illustrating an operation method when blood pressure is calculated according to an embodiment. FIG. 8 is a plan view illustrating a light emitting area PPA and a light sensing area PPB of the display device 1 according to the embodiment. FIG. 9 is a plan view illustrating the light emitting area PPA and the light sensing area PPB when first light and second light are emitted and sensed. FIG. 10 is a cross-sectional view taken along line II-II′ of FIG. 8.

Referring to FIGS. 8 through 10 in addition to FIG. 7, the light emitting area PPA and the light sensing area PPB may be disposed in a part of the active area AAR. In an embodiment, the light sensing area PPB may have a circular or oval shape, and the light emitting area PPA may have a donut or ring shape surrounding the light sensing area PPB, but the present disclosure is not limited thereto.

In the display device 1 according to an embodiment, the light emitting area PPA may surround the light sensing area PPB. In such an embodiment, since light reflected in various directions is received, the accuracy of blood pressure measurement may be improved.

The light emitting area PPA may refer to an area where the pixels PX are activated to emit light when blood pressure is measured. The light emitting area PPA may include one or more first pixels PX1 and one or more second pixels PX2. The light emitting area PPA may also include the third pixels PX3 and/or the fourth pixels PX4 together with the first pixels PX1 and the second pixels PX2. However, the present disclosure is not limited thereto, and the light emitting area PPA may also include various pixels as long as the light emitting area PPA includes the first pixels PX1 and the second pixels PX2.

The light sensing area PPB may refer to an area where the photosensors PS are activated to receive light when blood pressure is measured. The light sensing area PPB may include one or more photosensors PS. In an embodiment, the photosensors PS of the light sensing area PPB may sense light of different colors. In an embodiment, for example, one of the photosensors PS may sense red light, another of the photosensors PS may sense green light, and another of the photosensors PS may sense blue light. However, the present disclosure is not limited thereto, and alternatively, each of the photosensors PS may also sense light of all colors. That is, one photosensor PS may sense all of the light in various wavelength ranges.

When blood pressure is measured in the display device 1 according to an embodiment, either the first pixels PX1 or the second pixels PX2 of the light emitting area PPA may emit light. In an embodiment, for example, the first pixels PX1 and the second pixels PX2 may alternately emit light in a first unit section USEC1 (see FIG. 12) and a second unit section USEC2 (see FIG. 12) which will be described later. The photosensors PS of the light sensing area PPB may receive light reflected after being emitted from the first pixels PX1 and the second pixels PX2.

FIG. 9(a) illustrates a case where the first light is emitted when blood pressure is measured, and FIG. 9(b) illustrates a case where the second light is emitted when blood pressure is measured.

An area activated for light emission and sensing when the first light (e.g., green light) is emitted from the first pixels PX1 may be smaller than an area activated for light emission and sensing when the second light (e.g., red light) is emitted from the second pixels PX2.

In an embodiment, for example, as illustrated in FIGS. 9(a) and 9(b), a radius W1 of a light sensing area PPB_G when the first light is emitted from the first pixels PX1 may be smaller than a radius W3 of a light sensing area PPB_R when the second light is emitted from the second pixels PX2. In addition, a radius W2 of a light emitting area PPA_G when the first light is emitted from the first pixels PX1 may be smaller than a radius W4 of a light emitting area PPA_R when the second light is emitted from the second pixels PX2.

Since the wavelength of the first light emitted from the first pixels PX1 of a display layer DPL is shorter than the wavelength of the second light emitted from the second pixels PX2, first reflected light L1 of the first light emitted from the first pixels PX1 may be less diffracted than second reflected light L2 of the second light emitted from the second pixels PX2. Accordingly, the display device 1 according to an embodiment may control the area activated for light emission and sensing when the first light is emitted from the first pixels PX1 to be smaller than the area activated for light emission and sensing when the second light is emitted from the second pixels PX2, thereby increasing light reception efficiency.

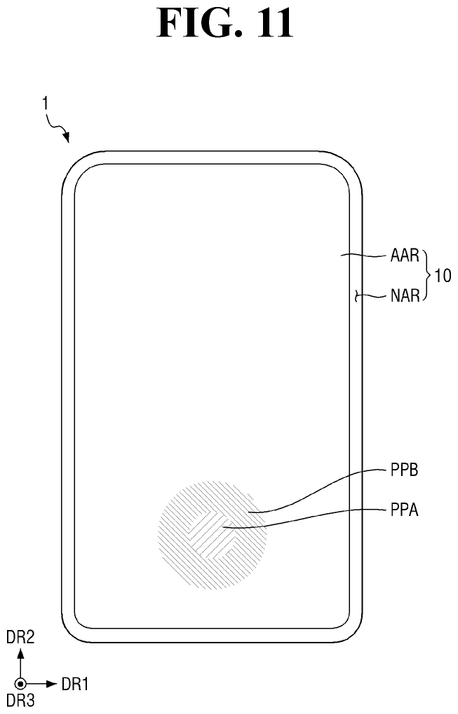

FIG. 11 is a plan view illustrating a light emitting area PPA and a light sensing area PPB of a display device 1 according to an embodiment. In description of the following embodiment, the same elements as those of the above-described embodiment will be indicated by the same reference numerals, and any repetitive detailed description thereof will be omitted or simplified, and differences will be mainly described.

Referring to FIG. 11, the display device 1 according to an embodiment is substantially the same as the display device 1 according to the embodiment described with reference to FIG. 8, etc. except that the light sensing area PPB surrounds the light emitting area PPA.

More specifically, in the display device 1 according to an embodiment, the light emitting area PPA may have a circular or oval shape, and the light sensing area PPB may have a donut or ring shape surrounding the light emitting area PPA, but the present disclosure is not limited thereto.

In the display device 1 according to an embodiment, since the light sensing area PPB surrounds the light emitting area PPA, the probability that light emitted from the light emitting area PPA will be received by the light sensing area PPB may be increased. Accordingly, the light reception efficiency may be improved.

FIG. 12 is a graph illustrating a first pulse wave signal PGS1 and a second pulse wave signal PGS2 according to an embodiment.

Referring to FIG. 12 in addition to FIG. 7, a photosensor PS may transmit a photocurrent signal LS generated by photocharges of incident reflected light L1 and L2 to the pulse wave sensing circuit 50. The photocurrent signal LS may include optical information.

The pulse wave sensing circuit 50 receiving the photocurrent signal LS including the optical information may generate a user's pulse wave signal based on the optical information. In an embodiment, for example, the pulse wave sensing circuit 50 may generate the first pulse wave signal PGS1 and the second pulse wave signal PGS2 respectively based on optical information of first reflected light L1 and optical information of second reflected light L2 sensed during a full section SEC. Then, the pulse wave sensing circuit 50 may provide the first pulse wave signal PGS1 and the second pulse wave signal PGS2 to the sensing determination unit 810 and the pulse wave correction unit 820 of the main processor 800.

The full section SEC may include a plurality of unit sections USEC. The unit sections USEC may include a plurality of first unit sections USEC1 including components of the first pulse wave signal PGS1 and a plurality of second unit sections USEC2 including components of the second pulse wave signal PGS2.

In an embodiment, the full section SEC may last (or have a time duration of) about 5 seconds to about 60 seconds. In an embodiment, for example, the full section SEC may last about 10 second to about 30 seconds. A unit section USEC may last about 0.1 second to about 3 seconds. In an embodiment, for example, the unit section USEC may last about 0.5 second to about 1.5 seconds.

The full section SEC may include a plurality of transition points TPT at which the first unit sections USEC1 and the second unit sections USEC2 are switched. The transition points TPT include first transition points TPT1 at which the first unit sections USEC1 are switched to the second unit sections USEC2 and second transition points TPT2 at which the second unit sections USEC2 are switched to the first unit sections USEC1.

In an embodiment, as illustrated in FIG. 12, the first unit sections USEC1 and the second unit sections USEC2 may be alternately located one by one. In an embodiment, for example, each of the first unit sections USEC1 may be a (2n−1)th section in the full section SEC, and each of the second unit sections USEC2 may be a (2n)th section in the full section SEC. Here, n may be a natural number equal to or greater than 1.

However, the present disclosure is not limited thereto. In an alternative embodiment, the first unit sections USEC1 and the second unit sections USEC2 may be alternately located while being repeated at least twice or more. In an embodiment, for example, the first unit sections USEC1 may be (4n−3)^{th} and (4n−2)^{th} sections in the full section SEC, and the second unit sections USEC2 may be (4n−1)^{th} and (4n)th sections in the full section SEC.

The first unit sections USEC1 and the second unit sections USEC2 may not necessarily be alternately located in the ratio of equal numbers and may also be alternately located in the ratio of different numbers as long as at least one transition point TPT is included during the full section SEC. In an embodiment, 5 to 20 transition points TPT may be included in the full section SEC to increase the accuracy of sensing measurement.

The first pulse wave signal PGS1 may include a plurality of first pulses PLS1 located in the first unit sections USEC1, respectively. The first pulse wave signal PGS1 may include pulses only in the first unit sections USEC1 and may not include pulses in the second unit sections USEC2.

The second pulse wave signal PGS2 may include a plurality of second pulses PLS2 located in the second unit sections USEC2, respectively. The second pulse wave signal PGS2 may include pulses only in the second unit sections USEC2 and may not include pulses in the first unit sections USEC1.

Each of the first and second pulses PLS1 and PLS2 may refer to a unit pulse generated during one unit section USEC.

The pulse end determination unit 811 of the sensing determination unit 810 receiving the first pulse wave signal PGS1 and the second pulse wave signal PGS2 may determine whether each of the first and second pulses PLS1 and PLS2 has ended. In an embodiment, for example, when amplitudes of the first and second pulses PLS1 and PLS2 reach a lowest point, the pulse end determination unit 811 may be determined that the first and second pulses PLS1 and PLS2 have ended. End points of the first and second pulses PLS1 and PLS2 may be the transition points TPT. The transition points TPT may be the lowest points of the first and second pulses PLS1 and PLS2.

In an embodiment, as shown in FIG. 7, when determining that each of the first and second pulses PLS1 and PLS2 has ended, the pulse end determination unit 811 may generate a pulse end signal PES and provide the pulse end signal PES to the sensing end determination unit 812.

The sensing end determination unit 812 receiving the pulse end signal PES may provide an emission start signal ESS1 or ESS2 to the first pixels PX1 or the second pixels PX2 and a sensing start signal SSS to the photosensors PS when the amplitude of a final pulse FPLS1 among the first pulses PLS1 or the amplitude of a final pulse FPLS2 among the second pulses PLS2 is greater than a threshold value THR.

When the amplitude of the final pulse FPLS1 among the first pulses PLS1 or the amplitude of the final pulse FPLS2 among the second pulses PLS2 is smaller than the threshold value THR, the sensing end determination unit 812 may provide emission end signals EES1 and EES2 to the first pixels PX1 and the second pixels PX2, provide a sensing end signal SES to the photosensors PS, and provide a correction start signal CSS to the pulse wave correction unit 820.

In the display device 1 according to an embodiment, since both the first pulse wave signal PGS1 and the second pulse wave signal PGS2 are sensed and generated during one full section SEC, measurement time may be reduced. In addition, since the first pulse wave signal PGS1 and the second pulse wave signal PGS2 are sensed and generated in the same time slot or during a same time period, a difference between information included in the signals can be minimized, which improves accuracy.

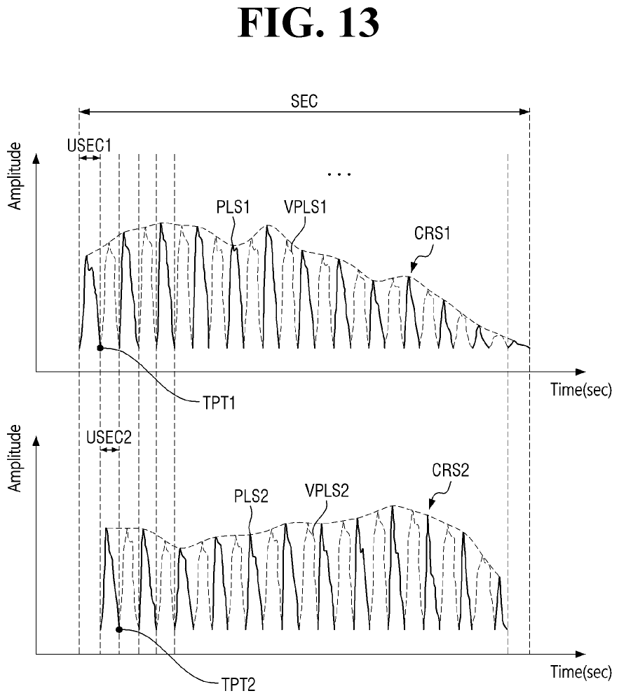

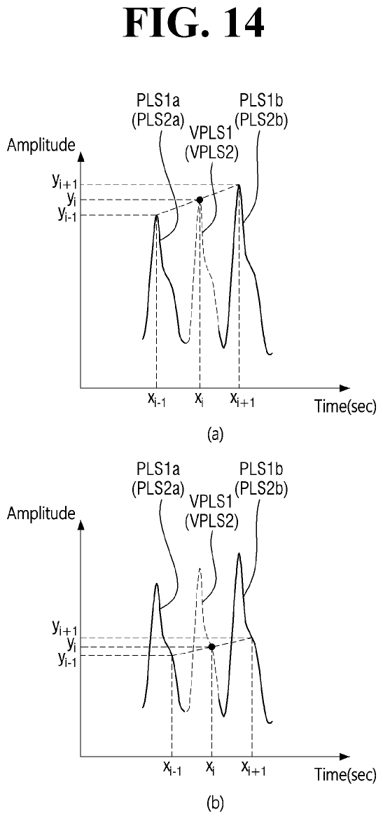

FIG. 13 is a graph illustrating a first correction signal CRS1 and a second correction signal CRS2 according to an embodiment. FIG. 14 is a graph illustrating a process of generating the first correction signal CRS1 and the second correction signal CRS2 according to an embodiment.

Referring to FIGS. 13 and 14 in addition to FIG. 7, the pulse wave correction unit 820 receiving the correction start signal CSS may generate the correction signals CRS1 through CRS3 by reconstructing and scaling the first pulse wave signal PGS1 and the second pulse wave signal PGS2.

The pulse wave correction unit 820 may include the first pulse wave correction unit 821 and the second pulse wave correction unit 822.

The first pulse wave correction unit 821 may generate the first correction signal CRS1 and the second correction signal CRS2 by reconstructing the first pulse wave signal PGS1 and the second pulse wave signal PGS2.

The first correction signal CRS1 and the second correction signal CRS2 may be signals obtained by adding first virtual pulses VPLS1 and second virtual pulses VPLS2 to the first pulse wave signal PGS1 and the second pulse wave signal PGS2, respectively. Each of the first virtual pulses VPLS1 and each of the second virtual pulses VPLS2 may be generated through a linear interpolation using arbitrary points included in at least two first pulses PLS1 adjacent to each other and arbitrary points included in at least two second pulses PLS2 adjacent to each other, respectively.

In an embodiment, a first point of each of the first virtual pulses VPLS1 is generated using a second point of a preceding pulse among the at least two first pulses adjacent to each other and a third point of a following (or subsequent) pulse among the at least two first pulses adjacent to each other.

In an embodiment, for example, referring to FIG. 14(a), coordinates of a peak point (or the second point) of a left pulse (or the preceding pulse) PLS1a or PLS2a are (xᵢ₋₁, yᵢ₋₁), and coordinates of a peak point (or the third point) of a right pulse (or a following pulse) PLS1b or PLS2b are (xᵢ₊₁, yᵢ₊₁). When coordinates of a peak point (or the first point) of a virtual pulse VPLS1 or VPLS2 are (xᵢ, yᵢ), the peak point of the virtual pulse VPLS1 or VPLS2 may satisfy Equation 1 below.

$${y}_{i} = \frac{{y}_{i + 1} - {y}_{i - 1}}{{x}_{i + 1} - {x}_{i - 1}} ( {x}_{i} - {x}_{i - 1} ) + {y}_{i - 1} . \qquad ( 1 )$$

Referring to FIG. 14(b), arbitrary points other than the peak point included in the virtual pulse VPLS1 or VPLS2 may also satisfy Equation 1 above.

In an embodiment, where the first unit sections USEC1 and the second unit section USEC2 are alternately located one by one, each of the first virtual pulses VPLS1 may be generated using an average value of the first pulses PLS1 located in a (2i−3)^{th} section and a (2i−1)^{th} section among the first unit sections USEC1, and each of the second virtual pulses VPLS2 may be generated using an average value of the second pulses PLS2 located in a (2j−2)^{th} section and a (2j)^{th} section among the second unit sections USEC2. Here, i and j may be natural numbers of 2 to n.

The first virtual pulses VPLS1 may be connected to the first transition points TPT1 of the first pulses PLS1, and the second virtual pulses VPLS2 may be connected to the second transition points TPT2 of the second pulses PLS2. Accordingly, each of the first correction signal CRS1 and the second correction signal CRS2 may be one continuous signal.

The first pulse wave correction unit 821 may provide the generated first and second corrected signals CRS1 and CRS2 to the second pulse wave correction unit 822.

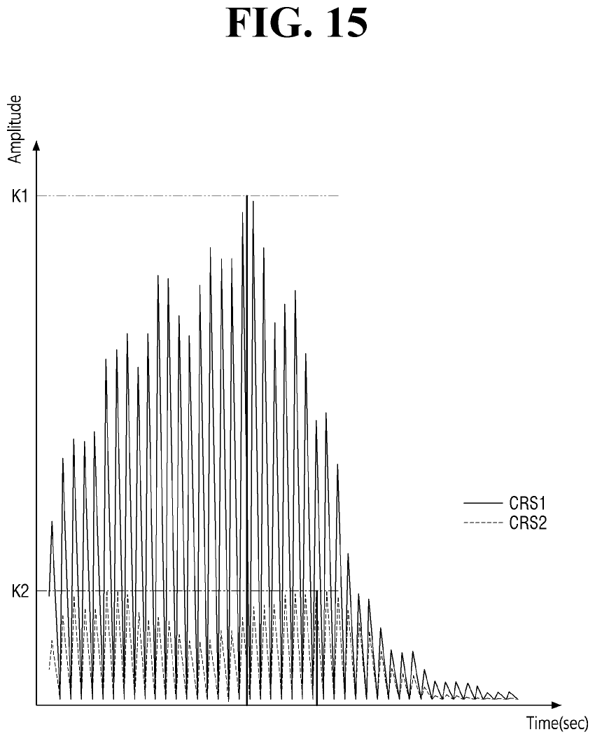

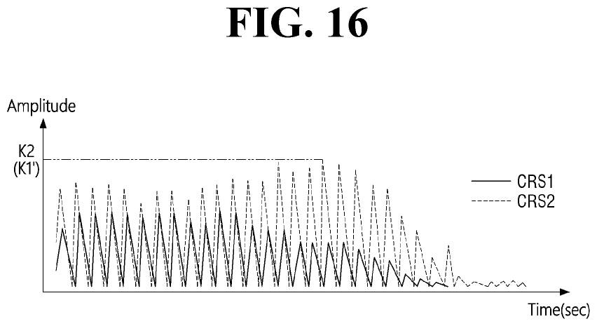

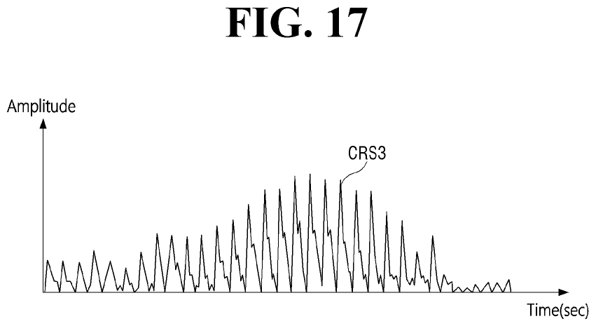

FIGS. 15 through 17 are graphs illustrating a process of generating a third correction signal CRS3 according to an embodiment.

Referring to FIGS. 15 through 17 in addition to FIG. 7, the second pulse wave correction unit 822 may generate the third correction signal CRS3 by removing noise from the second correction signal CRS2. In an embodiment, for example, the second pulse wave correction unit 822 may generate the third correction signal CRS3 by scaling the first correction signal CRS1 and removing the value of the scaled first correction signal CRS1 from the second correction signal CRS2.

The second pulse wave correction unit 822 receiving the first correction signal CRS1 and the second correction signal CRS2 may calculate a first maximum value K1 of the first correction signal CRS1 and a second maximum value K2 of the second correction signal CRS2.

The first maximum value K1 may be a largest signal value among signal values of the first correction signal CRS1. The second maximum value K2 may be a largest signal value among signal values of the second correction signal CRS2. Since the wavelength of light emitted from the first pixels PX1 is shorter than the wavelength of light emitted from the second pixels PX2, the first maximum value K1 of the first correction signal CRS1 generated by receiving reflected light L1 of the light emitted from the first pixels PX1 may be greater than the second maximum value K2 of the second correction signal CRS2 generated by receiving reflected light L2 of the light emitted from the second pixels PX2.

The second pulse wave correction unit 822 may scale the first correction signal CRS1 by using a ratio of the second maximum value K2 to the first maximum value K1. In an embodiment, for example, the second pulse wave correction unit 822 may reduce the magnitude of the first correction signal CRS1 by the ratio of the second maximum value K2 to the first maximum value K1. In this case, the magnitude of the scaled first correction signal CRS 1 may be generally smaller than that of the second correction signal CRS2. A maximum value K1′ of the scaled first correction signal CRS1 may be equal to the second maximum value K2.

The second pulse wave correction unit 822 may generate the third correction signal CRS3 by removing the value of the scaled first correction signal CRS1 from the second correction signal CRS2. Accordingly, the third correction signal CRS3 resulting from removal of noise components from the second correction signal CRS2 may be generated. In an embodiment, for example, when the first correction signal CRS1 is defined as CRS1, the first maximum value K1 is defined as K1, the second correction signal CRS2 is defined as CRS2, the second maximum value K2 is defined as K2, and the third correction signal CRS3 is defined as CRS3, the third correction signal CRS3 may be calculated by Equation 2 below.

$$CRS 3 = CRS 2 - \frac{K 2}{K 1} CRS 1. \qquad ( 2 )$$

In the display device 1 according to an embodiment, noise components present in the second correction signal CRS2 may be removed through the first correction signal CRS1. In an embodiment, for example, as described above, the second pulse wave signal PGS2 may include not only changes caused by arteries but also changes caused by capillaries as information, and the first pulse wave signal PGS1 may include changes caused by capillaries as information. Therefore, the first correction signal CRS1 generated from the first pulse wave signal PGS1 may be scaled and removed from the second correction signal CRS2 generated from the second pulse wave signal PGS2. As a result, in such an embodiment, noise may be effectively removed. Accordingly, the display device 1 according to an embodiment may accurately calculate blood pressure information.

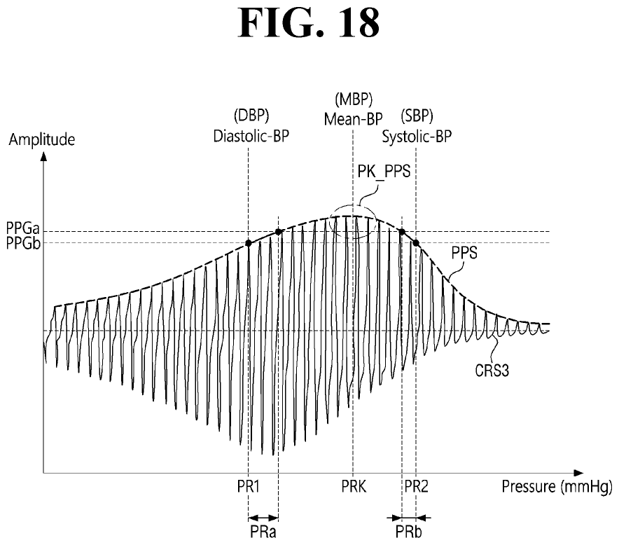

FIG. 18 is a graph illustrating a process of calculating blood pressure information BPD based on the third correction signal CRS3 according to an embodiment.

Referring to FIG. 18 in addition to FIG. 7, in an embodiment, the blood pressure calculation unit 830 may calculate the blood pressure information BPD based on the pressure measurement value PRM received from the pressure sensing circuit 40 and the third correction signal CRS3 received from the pulse wave correction unit 820.

The blood pressure calculation unit 830 may generate a peak detection signal PPS using peak values of the third correction signal CRS3. The peak detection signal PPS may be defined as a signal corresponding to a peak value of each cycle of the third correction signal CRS3. In an embodiment, for example, the third correction signal CRS3 may have one or more peak values. The blood pressure calculation unit 830 may calculate the peak detection signal PPS including points corresponding to the peak values of the third correction signal CRS3.

The blood pressure calculation unit 830 may calculate a first pressure measurement value PRK corresponding to a maximum value PK_PPS of the peak detection signal PPS. Based on the first pressure measurement value PRK, the blood pressure calculation unit 830 may calculate a mean blood pressure MBP equal to the first pressure measurement value PRK, a minimum blood pressure DBP lower than the first pressure measurement value PRK, a maximum blood pressure SBP higher than the first pressure measurement value PRK. The minimum blood pressure DBP may be diastolic blood pressure, and the maximum blood pressure SBP may be systolic blood pressure.

In an embodiment, the minimum blood pressure DBP may be defined as a minimum pressure measurement value PR1 among pressure measurement values PRa and PRb corresponding to values PPGb and PPGa corresponding to 60% to 80% of the maximum value PK_PPS of the peak detection signal PPS. The maximum blood pressure SBP may be defined as a maximum pressure measurement value PR2 among the pressure measurement values PRa and PRb corresponding to the values PPGb and PPGa corresponding to 60% to 80% of the maximum value PK_PPS of the peak detection signal PPS. However, the present disclosure is not limited to the range of 60% to 80%, and a different range may be applied for each user.

In the display device 1 according to an embodiment, since a pulse wave signal oscillates according to the heartbeat cycle, the pulse wave signal may reflect a change in blood pressure according to the heartbeat. In this case, since each user has a different heartbeat cycle and the pulse wave signal changes according to a change in the heartbeat cycle, each user may have a different pulse wave signal. Therefore, the display device 1 may effectively remove a different noise component for each user by generating the first pulse wave signal PGS1 and the second pulse wave signal PGS2 from light emitted from the first pixels PX1 and light emitted from the second pixels PX2, respectively. Accordingly, blood pressure may be accurately calculated.

A method of measuring blood pressure using a display device according to an embodiment will now be described. Any repetitive detailed description of the same elements and features as those described above will be omitted or simplified, and differences will be mainly described.

FIG. 19 is a flowchart illustrating a method of measuring blood pressure using a display device according to an embodiment.

Referring to FIG. 19, an embodiment of the method S1 of measuring blood pressure using the display device may include measuring a pressure measurement value according to a pressure applying time (operation S100), generating a first pulse wave signal and a second pulse wave signal (operation S200), generating a first correction signal and a second correction signal by reconstructing the first pulse wave signal and the second pulse wave signal, respectively (operation S300), generating a third correction signal by removing noise from the second correction signal based on the first correction signal and the second correction signal (operation S400), and calculating blood pressure information based on the third correction signal (operation S500).

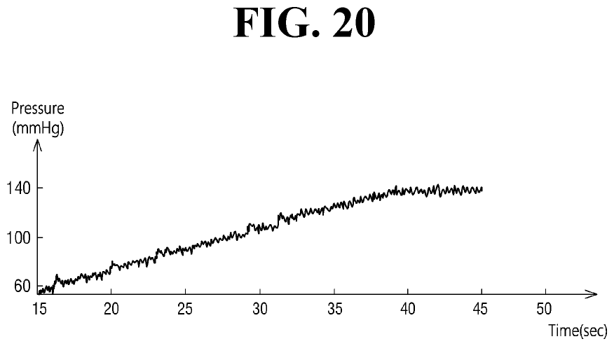

FIG. 20 is a graph illustrating the pressure measurement value in operation S100 according to an embodiment.

Referring to FIG. 20, in an embodiment, a pressure sensing circuit 40 may convert the pressure measured by a pressure sensor according to the pressure applying time into pressure measurement value information PRM. A user may apply pressure to a location where the pressure sensor is disposed, and the pressure sensor may transmit a resultant pressure change to the pressure sensing circuit 40 as an electrical signal.

The pressure sensing circuit 40 may measure and calculate a pressure measurement value PRM of the pressure applied by a user. In an embodiment, for example, when a user touches the display device 1 with a finger, the pressure measurement value PRM measured by the pressure sensor may gradually increase over time to reach a maximum value as illustrated in FIG. 20. As the pressure measurement value PRM (i.e., contact pressure) increases, the diameter of blood vessels may decrease, causing blood flow to decrease or become zero. The display device 1 according to an embodiment may calculate blood pressure information by tracking a change in blood volume according to the change in pressure. The change in blood volume may be included in a first pulse wave signal PGS1 and a second pulse wave signal PGS2.

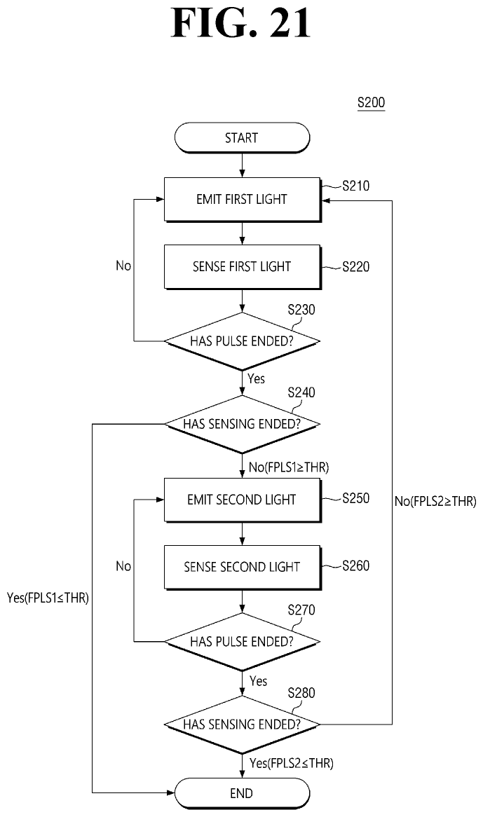

FIG. 21 is a flowchart illustrating operation S200 in FIG. 19 according to an embodiment. An embodiment in which first unit sections USEC1 and second unit sections USEC2 are alternately located one by one as illustrated in FIG. 12 will be described below as an example.

Referring to FIG. 21, in an embodiment, the generating of the first pulse wave signal and the second pulse wave signal (operation S200) may include a first light emitting operation (operation S210), a first light sensing operation (operation S220), a first pulse end determination operation (operation S230), a first sensing end determination operation (operation S240), a second light emitting operation (operation S250), a second light sensing operation (operation S260), a second pulse end determination operation (operation S270), and a second sensing end determination operation (operation S280).

In the first light emitting operation (operation S210), first pixels PX1 may emit first light.

In the first light sensing operation (operation S220), first reflected light L1 of the first light emitted from the first pixels PX1 may be sensed through photosensors PS. Information about the sensed first reflected light L1 may be provided to a pulse wave sensing circuit 50. The pulse wave sensing circuit 50 may generate a first pulse PLS1 of the first pulse wave signal PGS1 based on the information about the first reflected light L1.

In the first pulse end determination operation (operation S230), a pulse end determination unit 811 of a main processor 800 may determine whether the first pulse PLS1 has ended. When an end point of the first pulse PLS1, that is, a first transition point TPT1 is not reached, the pulse end determination unit 811 may continuously maintain light emission and sensing. When the first transition point TPT1 is reached, the pulse end determination unit 811 may provide a pulse end signal PES to a sensing end determination unit 812.

In the first sensing end determination operation (operation S240), the sensing end determination unit 812 of the main processor 800 may determine whether sensing has ended. When an amplitude of the first pulse PLS1 is greater than a threshold value THR, the sensing end determination unit 812 may provide an emission start signal ESS2 to second pixels PX2 and a sensing start signal SSS to the photosensors PS.

In the second light emitting operation (operation S250), the second pixels PX2 may emit second light.

In the second light sensing operation (operation S260), second reflected light L2 of the second light emitted from the second pixels PX2 may be sensed through the photosensors PS. Information about the sensed second reflected light L2 may be provided to the pulse wave sensing circuit 50. The pulse wave sensing circuit 50 may generate a second pulse PLS2 of the second pulse wave signal PGS2 based on the information about the second reflected light L2.

In the second pulse end determination operation (operation S270), the pulse end determination unit 811 of the main processor 800 may determine whether the second pulse PLS2 has ended. When an end point of the second pulse PLS2, that is, a second transition point TPT2 is not reached, the pulse end determination unit 811 may continuously maintain light emission and sensing. When the second transition point TPT2 is reached, the pulse end determination unit 811 may provide the pulse end signal PES to the sensing end determination unit 812.

In the second sensing end determination operation (operation S280), the sensing end determination unit 812 of the main processor 800 may determine whether sensing has ended. When an amplitude of the second pulse PLS2 is greater than the threshold value THR, the sensing end determination unit 812 may provide the emission start signal ESS1 to the first pixels PX1 and the sensing start signal SSS to the photosensors PS.

In such an embodiment, the operations from the first light emitting operation (operation S210) to the first sensing end determination operation (operation S240) or from the first light emitting operation (operation S210) to the second sensing end determination operation (operation S280) may be repeated. As the above processes are repeated during a full section SEC, the first pulse wave signal PGS1 and the second pulse wave signal PGS2 may respectively include a plurality of first pulses PLS1 and a plurality of second pulses PLS2 respectively located in the alternating first and second unit sections USEC1 and USEC2.

In the first sensing end determination operation (operation S240) or the second sensing end determination operation (operation S280), the sensing end determination unit 812 of the main processor 800 may end sensing when an amplitude of a final pulse FPLS1 among the first pulses PLS1 or an amplitude of a final pulse FPLS2 among the second pulses PLS2 is smaller than the threshold value THR.

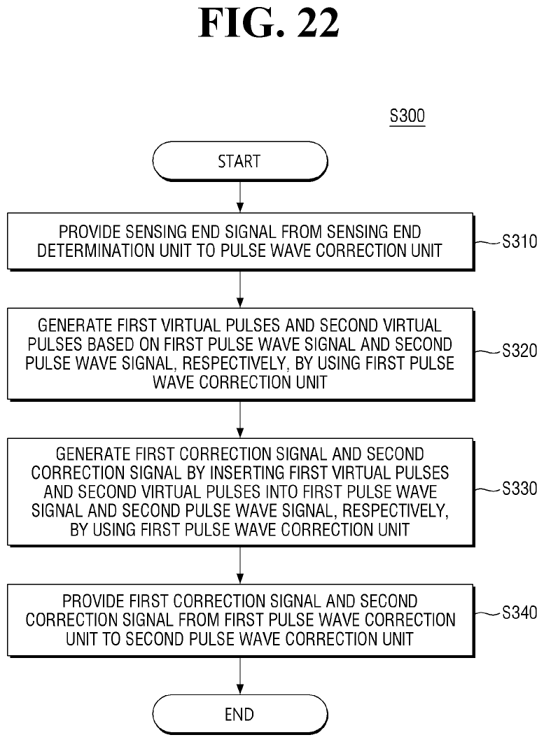

FIG. 22 is a flowchart illustrating operation S300 in FIG. 19 according to an embodiment.

Referring to FIG. 22, in an embodiment, the generating of the first correction signal and the second correction signal by reconstructing the first pulse wave signal and the second pulse wave signal, respectively, (operation S300) may include providing a sensing end signal from a sensing end determination unit to a pulse wave correction unit (operation S310), generating first virtual pulses and second virtual pulses based on the first pulse wave signal and the second pulse wave signal, respectively, by using the first pulse wave correction unit (operation S320), generating the first correction signal and the second correction signal by inserting the first virtual pulses and the second virtual pulses into the first pulse wave signal and the second pulse wave signal, respectively, by using the first pulse wave correction unit (operation S330), and providing the first correction signal and the second correction signal from the first pulse wave correction unit to a second pulse wave correction unit (operation S340).

In the providing of the sensing end signal from the sensing end determination unit to the pulse wave correction unit (operation S310), the sensing end determination unit 812 may provide a sensing end signal SES to a pulse wave correction unit 820.

In the generating of the first virtual pulses and the second virtual pulses (operation S320), a first pulse wave correction unit 821 may generate first virtual pulses VPLS1 and second virtual pulses VPLS2 based on the first pulse wave signal PGS1 and the second pulse wave signal PGS2, respectively.

In the generating of the first correction signal and the second correction signal (operation S330), the first pulse wave correction unit 821 may generate a first correction signal CRS1 and a second correction signal CRS2 by inserting the first virtual pulses VPLS1 and the second virtual pulses VPLS2 into the first pulse wave signal PGS1 and the second pulse wave signal PGS2, respectively.

Since the method of generating the first correction signal and the second correction signal by inserting the first virtual pulses VPLS1 and the second virtual pulses VPLS2 are substantially the same as that described above, any repetitive detailed description thereof will be omitted.

In the providing of the first correction signal and the second correction signal from the first pulse wave correction unit to the second pulse wave correction unit (operation S340), the first pulse wave correction unit 821 may provide the first correction signal CRS1 and the second correction signal CRS2 to the second pulse wave correction unit 822.

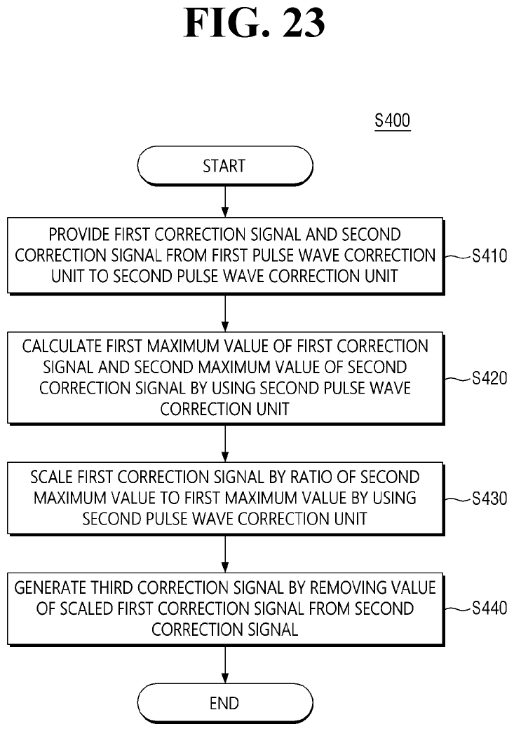

FIG. 23 is a flowchart illustrating operation S400 in FIG. 19 according to an embodiment.

Referring to FIG. 23, the generating of the third correction signal by removing noise from the second correction signal based on the first correction signal and the second correction signal (operation S400) may include providing the first correction signal and the second correction signal from the first pulse wave correction unit to the second pulse wave correction unit (operation S410), calculating a first maximum value of the first correction signal and a second maximum value of the second correction signal by using the second pulse wave correction unit (operation S420), scaling the first correction signal by using the second pulse wave correction unit (operation S430), and generating the third correction signal by removing a value of the scaled first correction signal from the second correction signal using the second pulse wave correction unit (operation S440).

In the providing of the first correction signal and the second correction signal from the first pulse wave correction unit to the second pulse wave correction unit (operation S410), the first pulse wave correction unit 821 may provide the first correction signal CRS1 and the second correction signal CRS2 to the second pulse wave correction unit 822.

In the calculating of the first maximum value of the first correction signal and the second maximum value of the second correction signal by using the second pulse wave correction unit (operation S420), the second pulse wave correction unit 822 may calculate a first maximum value K1 of the first correction signal CRS1 and a second maximum value K2 of the second correction signal CRS2.

In the scaling of the first correction signal by using the second pulse wave correction unit (operation S430), the second pulse wave correction unit 822 may scale the first correction signal CRS1 by using a ratio of the second maximum value K2 to the first maximum value K1.

In the generating of the third correction signal by removing the value of the scaled first correction signal from the second correction signal using the second pulse wave correction unit (operation S440), the second pulse wave correction unit 822 may generate a third correction signal CRS3 by removing the value of the scaled first correction signal CRS1 from the second correction signal CRS2.

Since the method of generating the third correction signal CRS3 by calculating the first maximum value K1 of the first correction signal CRS1 and the second maximum value K2 of the second correction signal CRS2 and scaling the first correction signal CRS1 is substantially the same as that described above, any repetitive detailed description thereof will be omitted.

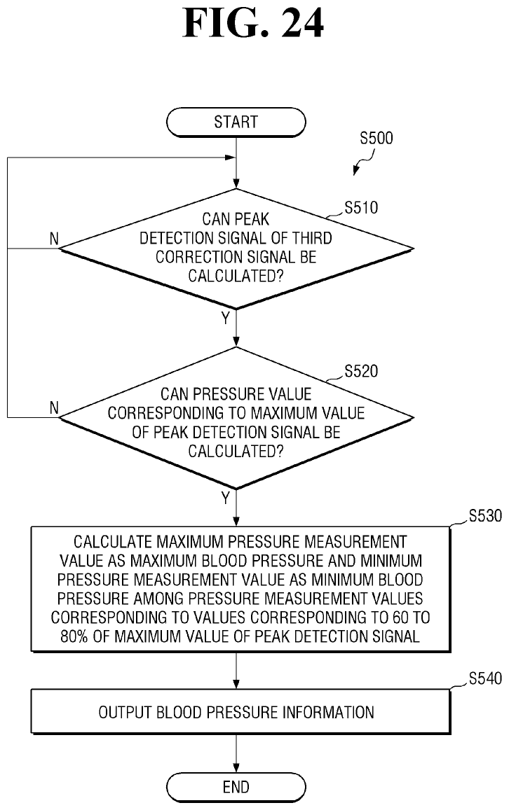

FIG. 24 is a flowchart illustrating operation S500 in FIG. 19 according to an embodiment.

Referring to FIG. 24, the calculating of the blood pressure information based on the third correction signal (operation S500) may include determining whether a peak detection signal of the third correction signal can be calculated (operation S510), determining whether a pressure value corresponding to a maximum value of the peak detection signal can be calculated (operation S520), calculating a maximum pressure measurement value as maximum blood pressure and a minimum pressure measurement value as minimum blood pressure among pressure measurement values corresponding to values corresponding to 60% to 80% of the maximum value of the peak detection signal (operation S530), and outputting blood pressure information (operation S540).

In the determining of whether the peak detection signal of the third correction signal can be calculated (operation S510), a blood pressure calculation unit 830 may generate a peak detection signal PPS using peak values of the third correction signal CRS3.

In the determining of whether the pressure value corresponding to the maximum value of the peak detection signal can be calculated (operation S520), when a maximum value PK_PPS of the peak detection signal PPS exists, the blood pressure calculation unit 830 may calculate a first pressure measurement value PRK corresponding to the maximum value PK_PPS of the peak detection signal PPS.

In the calculating of the maximum pressure measurement value as the maximum blood pressure and the minimum pressure measurement value as the minimum blood pressure among the pressure measurement values corresponding to the values corresponding to 60% to 80% of the maximum value of the peak detection signal (operation S530), the blood pressure calculation unit 830 may calculate mean blood pressure MBP equal to the first pressure measurement value PRK, minimum blood pressure DBP lower than the first pressure measurement value PRK, and maximum blood pressure SBP higher than the first pressure measurement value PRK based on the first pressure measurement value PRK. As described above, the present disclosure is not limited to the range of 60% to 80%, and a different range may be applied for each user.

In the outputting of the blood pressure information (operation S540), the blood pressure calculation unit 830 may output blood pressure information BPD through a display panel 10.

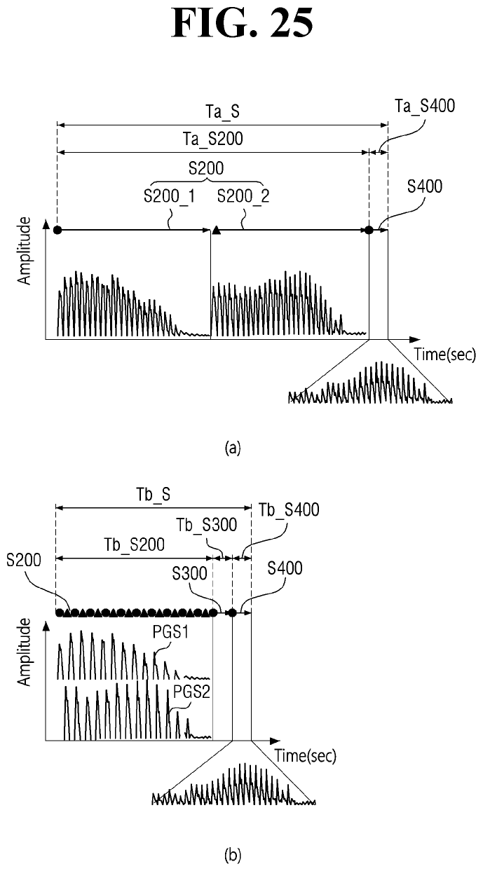

FIG. 25 is a graph comparing measurement times in methods of measuring blood pressure using a conventional display device and a display device according to an embodiment. FIG. 25(a) illustrates a method of measuring blood pressure using a conventional display device, and FIG. 25(b) illustrates a method of measuring blood pressure using a display device according to an embodiment.

Referring to FIG. 25, in the method of measuring blood pressure using the conventional display device, the generating of the first pulse wave signal and the second pulse wave signal (operation S200) includes generating the first pulse wave signal (operation S200_1) and generating the second pulse wave signal (operation S200_2) in two full sections SEC, respectively. The method of measuring blood pressure using the conventional display device may not include the generating of the first correction signal and the second correction signal (operation S300), and the generating of the third correction signal (operation S400) may be performed immediately thereafter.

In the method of measuring blood pressure using the display device according to an embodiment, the generating of the first pulse wave signal and the second pulse wave signal (operation S200) may be performed simultaneously during one full section SEC. In the method of measuring blood pressure using the display device according to an embodiment, the generating of the first correction signal and the second correction signal (operation S300) may be performed, and then the generating of the third correction signal (operation S400) may be performed.

Accordingly, a first used time Ta_S200 in the generating of the first pulse wave signal and the second pulse wave signal (operation S200) in the method of measuring blood pressure using the conventional display device may be longer than a second used time Tb_S200 in the generating of the first pulse wave signal and the second pulse wave signal (operation S200) in the method of measuring blood pressure using the display device according to the current embodiment. In an embodiment, for example, the first used time Ta_S200 may be twice as long as the second required time Tb_S200. In some embodiments, the second used time Tb_S200 may be about 5 seconds to about 60 seconds.

Even if the method of measuring blood pressure using the display device according to an embodiment includes the generating of the first correction signal and the second correction signal (operation S300), since the time used for the generating of the first correction signal and the second correction signal (operation S300) is only about 0.1 second to several seconds, the total time used Tb_S in the method of measuring blood pressure using the display device according to an embodiment may be shorter than the total time used Ta_S in the method of measuring blood pressure using the conventional display device.

In addition, in the method of measuring blood pressure using the display device according to an embodiment, since the generating of the first pulse wave signal and the second pulse wave signal (operation S200) is performed simultaneously during one full section SEC, the first pulse wave signal PGS1 and the second pulse wave signal PGS2 are sensed in the same time slot. Therefore, a difference between information included in the first and second pulse wave signals can be minimized, which improves accuracy.

The invention should not be construed as being limited to the embodiments set forth herein. Rather, these embodiments are provided so that this disclosure will be thorough and complete and will fully convey the concept of the invention to those skilled in the art.

While the invention has been particularly shown and described with reference to embodiments thereof, it will be understood by those of ordinary skill in the art that various changes in form and details may be made therein without departing from the spirit or scope of the invention as defined by the following claims.

What is claimed is:

1. A display device comprising:
a first pixel which emits first light;
a second pixel which emits second light having a different wavelength from the first light;
a photosensor which senses the first light and the second light which are alternately provided a plurality of times; and
a pulse wave sensing circuit which receives information about the first light and information about the second light sensed by the photosensor and generates a first pulse wave signal based on the information about the first light and a second pulse wave signal based on the information about the second light; and
a main processor which receives the first pulse wave signal and the second pulse wave signal and calculates blood pressure information.

2. The display device of claim 1, wherein
the first pulse wave signal and the second pulse wave signal are respectively generated based on the first light and the second light alternately sensed in one full section, and
the one full section comprises:
a plurality of first unit sections comprising components of the first pulse wave signal;
a plurality of second unit sections comprising components of the second pulse wave signal; and
a plurality of transition points at which the first unit sections and the second unit sections are switched.

3. The display device of claim 2, wherein
the first pulse wave signal comprises a plurality of first pulses located in the first unit sections,
the second pulse wave signal comprises a plurality of second pulses located in the second unit sections, and
the main processor generates
a first correction signal by inserting first virtual pulses, each of which is generated based on at least two first pulses adjacent to each other, into the second unit sections of the first pulse wave signal, and
a second correction signal by inserting second virtual pulses, each of which is generated based on at least two second pulses adjacent to each other, into the first unit sections of the second pulse wave signal.

4. The display device of claim 3, wherein
each of the first virtual pulses is generated through a linear interpolation using arbitrary points included in the at least two first pulses adjacent to each other, and
each of the second virtual pulses is generated through a linear interpolation using arbitrary points included in the at least two second pulses adjacent to each other.

5. The display device of claim 4, wherein
a first point of each of the first virtual pulses is generated using a second point of a preceding pulse among the at least two first pulses adjacent to each other and a third point of a following pulse among the at least two first pulses adjacent to each other,
coordinates of the first point are (xᵢ, yᵢ),
coordinates of the second point are (xᵢ₋₁, yᵢ₋₁),
coordinates of the third point are (xᵢ₊₁, yᵢ₊₁), and
the first point of each of the first virtual pulses satisfies the following equation:
$${y}_{i} = \frac{{y}_{i + 1} - {y}_{i - 1}}{{x}_{i + 1} - {x}_{i - 1}} ( {x}_{i} - {x}_{i - 1} ) + {y}_{i - 1} .$$

6. The display device of claim 3, wherein
each of the first unit sections is a (2n−1)-th section in the one full section,
each of the second unit sections is a (2n)-th section in the one full section, and
n is a natural number equal to or greater than 1.

7. The display device of claim 6, wherein
each of the first virtual pulses is generated using an average value of the first pulses located in a (2i−3)-th section and a (2i−1)-th section among the first unit sections,
each of the second virtual pulses is generated using an average value of the second pulses located in a (2j−2)-th section and a (2j)-th section among the second unit sections, and
i and j are natural numbers of 2 to n.

8. The display device of claim 3, wherein the transition points are lowest points of the first pulses and the second pulses, respectively.

9. The display device of claim 3, wherein the one full section ends when an amplitude of a final pulse among the first pulses or an amplitude of a final pulse among the second pulses is smaller than a threshold value.

10. The display device of claim 3, wherein
the main processor generates a third correction signal by removing noise from the second correction signal based on the first correction signal and the second correction signal, and
the third correction signal is generated based on a ratio of a first maximum value of the first correction signal and a second maximum value of the second correction signal.

11. The display device of claim 10, wherein when
the first correction signal is defined as CRS1,
the first maximum value is defined as K1,
the second correction signal is defined as CRS2,
the second maximum value is defined as K2, and
the third correction signal is defined as CRS3,
wherein the third correction signal is calculated by the following equation:
$$CRS 3 = CRS 2 - \frac{K 2}{K 1} CRS 1.$$

12. The display device of claim 10, further comprising:
a pressure sensor which senses a pressure applied from an outside,
wherein the main processor
calculates blood pressure information based on a pressure measurement value measured by the pressure sensor and the third correction signal,
generates a peak detection signal by connecting peak values of the third correction signal,
calculates a first pressure measurement value corresponding to a maximum value of the peak detection signal, and
calculates mean blood pressure equal to the first pressure measurement value, minimum blood pressure lower than the first pressure measurement value, and maximum blood pressure higher than the first pressure measurement value based on the first pressure measurement value.

13. The display device of claim 12, wherein
the minimum blood pressure is defined as a minimum pressure measurement value among pressure measurement values corresponding to values corresponding to 60% to 80% of the maximum value of the peak detection signal, and
the maximum blood pressure is defined as a maximum pressure measurement value among the pressure measurement values corresponding to the values corresponding to 60% to 80% of the maximum value of the peak detection signal.

14. The display device of claim 1, wherein a wavelength of the first light is smaller than a wavelength of the second light.

15. The display device of claim 14, wherein
the first light is green light or blue light, and
the second light is red light or infrared light.

16. The display device of claim 1, further comprising:
a display panel comprising the first pixel, the second pixel, and the photosensor, wherein
the display panel comprises a first light sensing area in which the first light is sensed and a second light sensing area in which the second light is sensed, and
the first light sensing area is smaller in size than the second light sensing area in a plan view.

17. The display device of claim 16, wherein
the first and second light sensing areas are surrounded by the first pixel and the second pixel, respectively, and
a first light emitting area in which the first pixel emits light is larger in size than a second light emitting area in which the second pixel emits light.

18. A method of measuring blood pressure using a display device which comprises a first pixel which emits first light, a second pixel which emits second light having a different wavelength from the first light, a photosensor which senses the first light and the second light which are alternately provided a plurality of times, a pulse wave sensing circuit which receives information about the first light and information about the second light sensed by the photosensor and generates a first pulse wave signal based on the information about the first light and a second pulse wave signal based on the information about the second light, a pressure sensor which senses a pressure applied from an outside, and a main processor which receives the first pulse wave signal and the second pulse wave signal and calculates blood pressure information, the method comprising:
measuring a pressure measurement value according to a pressure applying time through the pressure sensor;
generating a correction signal based on the first pulse wave signal and the second pulse wave signal; and
calculating blood pressure information based on the pressure measurement value and the correction signal.

19. The method of claim 18, wherein the generating the correction signal based on the first pulse wave signal and the second pulse wave signal comprises:
generating first virtual pulses and second virtual pulses based on the first pulse wave signal and the second pulse wave signal, respectively; and
generating a first correction signal and a second correction signal by inserting the first virtual pulses and the second virtual pulses into the first pulse wave signal and the second pulse wave signal, respectively.

20. The method of claim 19, wherein
each of the first virtual pulses is generated through a linear interpolation using arbitrary points included in at least two first pulses adjacent to each other among a plurality of first pulses of the first pulse wave signal, which is sensed in one full section, and
each of the second virtual pulses is generated through a linear interpolation using arbitrary points included in at least two second pulses adjacent to each other among a plurality of second pulses of the second pulse wave signal, which is sensed in the one full section.
